(19) United States
(12) Patent Application Publication
(10) Pub. No.: US 2012/0142603 A1
(43) Pub. Date: Jun. 7, 2012

(54) BIOADHESIVE PATCH FOR SUTURELESS CLOSURE OF SOFT TISSUE

(75) Inventors: Francis S. Markland, Manhattan Beach, CA (US); Stephen D. Swenson, Arcadia, CA (US); Radu O. Minea, Arcadia, CA (US)

(73) Assignee: University of Southern California

(21) Appl. No.: 13/201,433

(22) PCT Filed: Feb. 12, 2010
(86) PCT No.: PCT/US10/24166

§ 371 Date: Feb. 9, 2012

Related U.S. Application Data
(60) Provisional application No. 61/152,235, filed on Feb. 12, 2009.
(60) Provisional application No. 61/303,631, filed on Feb. 11, 2010.

(51) Int. Cl. A61K 38/02; C12N 5/0797; C12N 5/071

(52) U.S. Cl. 514/17.7; 514/20.8; 514/1.1; 435/395

(57) ABSTRACT

Provided herein are compositions and methods for treating soft tissue injuries using a patch having a polymer on its surface linked to polypeptides having a disintegrin domain. The polypeptides having a disintegrin domain can include contortrostatin, vicrostatin, and ADAM derived polypeptides. Compositions of the invention can be used for the treatment of injuries to soft tissues that include the eye, liver and brain.

### CROSS-REFERENCE TO RELATED APPLICATIONS

This application claims the benefit of priority under 35 U.S.C. §119(e) to U.S. Provisional Application Ser. No. 61/152,235, filed Feb. 12, 2009, and to U.S. Provisional Application Ser. No. 61/303,631, filed Feb. 11, 2010, which are incorporated by reference herein in their entirety including all figures and tables.

### FIELD OF INVENTION

The invention relates to methods of treating soft tissue injuries using a patch having a polymer on its surface linked to polypeptides comprising a disintegrin domain. Such polypeptides include contortrostatin, vicrostatin, and ADAM derived polypeptides. The invention relates to the treatment of injuries to soft tissues that include the eye, liver and brain.

### BACKGROUND OF THE INVENTION

The invention is related to U.S. patent Ser. No. 11/351,311 by Minea et al., and titled “Method of expressing proteins with disulfide bridges,” to PCT Patent Application No. PCT/US09/64256, filed Nov. 12, 2009, and titled “Method of expressing proteins with disulfide bridges with enhanced yields and activity,” and U.S. Publication no. 20080306611 by Rowley et al., and titled “Biocompatible implants and methods of making and attaching same.” The contents of all are incorporated herein by reference thereto including all figures.

The U.S. Army and other branches of the U.S. military fight wars not only to keep the homeland free, but also to protect the local population in various parts of the world. With the changing scenario in the war zone, the way battles are fought has changed over the years. Unfortunately, what has not changed is the likelihood of trauma during battle. Rapid availability of advanced medical and surgical care for injured soldiers on the battlefront has reduced the rates of mortality. However, with the new type of insurgencies around the world and the use of IEDs (improvised explosive devices), the incidence and severity of injury is increasing, especially in the field of soft tissue trauma. For example, severely traumatized eyes were considered non-salvageable and were enucleated. Other non-suturable tissues include liver or brain.

Integrins are heterodimers composed of alpha and beta submits that are non-covalently associated. Interactions between integrins and ECM proteins have been shown to be mediated via an Arg-Gly-Asp (RGD) sequence present in the matrix proteins. Both the alpha and beta subunits of the integrin are required for ECM protein binding.

A well known inhibitor of the integrin-ECM interaction is a disintegrin which represents a family of proteins that include those from venom of snakes of the Crotalidae and Viperidae. Disintegrin families have been found to inhibit glycoprotein (GP) IIb/IIIa mediated platelet aggregation. Disintegrins are disulfide rich and many contain an RGD (Arg-Gly-Asp) sequence that has been implicated in the inhibition of integrin-mediated interactions.

The RGD sequence of a polypeptide comprising a disintegrin domainis located at the tip of a flexible loop, the integrin-binding loop, stabilized by disulfide bonds and protruding from the main body of the polypeptide chain. This exposed RGD sequence enables polypeptides comprising a disintegrin domain to bind to integrins with high affinity.

Polypeptides comprising a disintegrin domain that are known to disrupt integrin interactions include bitistatin, an 83 amino acid disintegrin isolated from the venom of Bitis arietans; echistatin, a 49 amino acid disintegrin isolated from the venom of Echis cannatus; kistrin, a 68 amino acid disintegrin isolated from the venom of Calloselasma rhodostoma; trigamin, a 72 amino acid disintegrin isolated from the venom of Trimeresurus gramineus; applaggin, isolated from the venom of Agkistrodon piscivorus piscivorus; and contortrostatin (CN), isolated from the venom of Agkistrodon contortix contortix (the southern copperhead snake).

CN full-length DNA precursor has been cloned and sequenced [1] and the sequence can be accessed in the GenBank database using accession number: AF212305. CN is produced in the snake venom gland as a multidomain precursor of 2027 bp having a 1449 bp open reading frame encoding a precursor that includes a pro-protein domain (amino acid residues 1 to 190 of SEQ ID NO: 1), a metalloproteinase domain (residues 191 to 410 of SEQ ID NO: 1) and a disintegrin domain (residues 419 to 483 of SEQ ID NO: 1).

Receptors of CN that have been identified include: integrins αIIbβ3, αvβ3, αvβ5, and α5β1

U.S. patent Ser. No. 11/351,311 describes vicrostatin (VCN), a recombinant fusion protein wherein the last three amino acids of the carboxy terminus of CN are swapped with the C-terminal tail of echistatin (HGKPAT), and its expression in the Origami B (DE3)/pET32a system. Unlike other E. coli strains, the Origami B is unique in that, by carrying mutations in two key genes, thioredoxin reductase (trxB) and glutathione reductase (gor), that are critically involved in the control of the two major oxido-reductive pathways in E. coli, this bacterium cytoplasmic microenvironment is artificially shifted to a more oxidative redox state, which is the catalyst state for disulfide bridge formation in proteins. An improved method of expression of VCN is disclosed in PCT Patent Application No. PCT/US09/64256.

Other polypeptides comprising a disintegrin domain include ADAMs (A Disintegrin and Metalloproteinase). There are over 30 ADAM proteins indentified in the mammalian kingdom (of which humans possess 20 genes and 3 pseudogenes) and all of them include polypeptides comprising a disintegrin domain.

US Patent Publication No. 20080306611 describes the tacking of a laser-activated silicone prosthesis coated with at least one compound capable of binding to one or more integrins.

### SUMMARY OF THE INVENTION

Provided herein are methods for treating a soft tissue injury in an individual by applying to the surface of the soft tissue injury a patch containing a polymer on its surface linked to polypeptides with a disintegrin domain which facilitate attachment of the patch to the site of the soft tissue injury, thereby treating the soft tissue injury. The polymer can be silicone with an activated surface. The polymer can be a parylene. The methods include polypeptides such as contortrostatin (CN), vicrostatin (VCN), APs or MAPs. The MAPs can be MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32, or MAP33. The soft tissue can be eye, the liver, or brain cortex. Structures of the eye can be retina, conjunctiva, cornea, sclera, lens or choroid. The patch can be reversibly bound to the soft tissue injury site. The patch can further include a coating of one or more drugs.

Provided herein are also methods for growing cells by contacting the cells with a patch containing a polymer on its surface linked to polypeptides having a disintegrin domain that facilitate attachment of the cells to the patch, thereby aiding growth of the cells. The polymer can be silicone with an activated surface. The polymer can be a parylene. The methods include polypeptides such as contortrostatin (CN), vicrostatin (VCN), APs or MAPs. The MAPs can be MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32, or MAP33. The cells can be stem cells.

### DESCRIPTION OF THE FIGURES

FIG. 1 Shows a comparison of polypeptides comprising a disintegrin domain of PIII-class snake venom metalloproteases (VAP1 and catrocollastatin) aligned with the polypeptides comprising a disintegrin domain of long-sized snake venom disintegrins (salmosin3 and bitistatin), the prototypical medium-sized snake venom disintegrin (trimestatin) as well as polypeptides comprising a disintegrin domain from human ADAM-derived Polypeptide (AP) in order to illustrate the rationale behind the MAP design. The structural elements generally present in polypeptides comprising a disintegrin domain of PIII-SVMPs and APs that are modified for the disintegrin domain to adopt the disintegrin fold of snake venom disintegrins are stricken through. The portion of the former spacer region that existed between the metalloprotease and disintegrin domains in the precursors of the long-sized disintegrins (e.g., bitistatin, salmosin3) and is released together with their disintegrin domains is depicted in bold.

FIG. 2 shows the alignment of selected native human AP disintegrin domains and highlights the residues that are modified (stricken through ) in MAP constructs. Additionally, in two cases (the ADAM disintegrin domains 1 and 17 or AP1 and AP17) a native residue (bold and double-underlined) was replaced with another amino acid according to the general cysteine pattern of these artificial MAPs. The tripeptide motif located at the tip of the disintegrin loop is highlighted in a [symbol] . The amino acid residues that make the disintegrin loop in each AP are italicized.

FIG. 3 shows select MAP sequences aligned with trimestatin, a prototypical medium-size snake venom disintegrin. In the sequences shown all cysteine residues are depicted in black underline whereas the tripeptide motif at the tip of disintegrin loops in trimestatin and MAPs are in a [symbol] .

FIGS. 4A-4F show a listing of MAP DNA sequences that were cloned into pET32a expression vector.

FIGS. 5A and 5B show a listing of oligonucleotide primers utilized for MAPs cloning into the pET32a vector.

FIGS. 6A-6H show the amino acid sequences of TrxA-MAP constructs that were expressed in Origami B (DE3). TrxA is thioredoxin A. The active site of TrxA and the tripeptide motif at the tip of the disintegrin loop are underlined, the TEV protease cleavage site is highlighted in a [symbol] and the linker region between TrxA and various MAP constructs is in bold black and italicized. The new residues introduced to replace the native residues in MAPs 1 and 17 are highlighted in bold double-underlined.

### DETAILED DESCRIPTION OF THE INVENTION

Provided herein is a patch containing a polymer with a surface linked with a polypeptide containing a disintegrin domain. The polymer can be silicone that has at least one activated surface formed by irradiation with laser light at a wavelength and power sufficient to eject organic species from the silicone substrate. As discussed below, other polymers can be used.

Nonlimiting examples of silicone patches include silicone films, substrates, bulk objects, and silicone coatings. The silicone may be present as substantially pure silicone polymers or, more typically, silicone polymers containing one or more additives to enhance the article's mechanical, thermal, or other physical characteristics. Nonlimiting examples of such additives include fillers, such as silica entities (e.g., foamed, granular, fibrous, etc; optionally, the silicone polymers are coupled to these silica entities via grafting), plasticizers, and crosslinkers, which can be admixed with the silicone-silica compounds to ensure lateral coupling between polymeric chains that are attached (i.e. grafted) to the same silica piece; etc. The whole of a silicone/silica/crosslinker assembly constitutes a silicone rubber. Varying any of the individual constituents in quality and quantity provides a nearly infinite range of silicone rubbers that can be activated according to the invention.

Integrins are integral membrane proteins used by cells to attach to their extracellular environment. In an embodiment, treating an activated silicone surface with a compound capable of binding to one or more integrins makes it possible to attach a silicone patch directly to injured tissue, without resort to surgical tacks, toxic adhesives, or other potentially destructive means.

To prepare a silicone patch having at least one activated surface, laser light of sufficient wavelength and power is directed at one or more surfaces of the silicone patch, which causes chemical bond breaking and formation of unpaired electrons, as described below. This “activates” the surface of the silicone patch in and around the areas that have been irradiated, making that area more chemically reactive toward other compounds.

The use of a monochromatic, intense UV light source can, under specific conditions, allow substantially instant light absorption and drive the silicone structure to destabilize its atomic configuration. This can be achieved with a laser source working in the UV range and under a pulsed regime. A suitable such laser is an excimer laser.

After investigating the actual optical absorption of a given silicone or silicone rubber, a UV light wavelength (or photon energy) is chosen that allows the material to absorb the UV photons selectively and exclusively on the Si—C bond electrons. Above a given power of the light source at that wavelength (of the order of 100 MW), all Si—C bond electrons that are present in the silicone volume that is traversed by the laser beam may be brought to absorb these UV photons quasi-simultaneously, over a very short period of time (on the order of 1-2 ns). That absorption produces the quasi-simultaneous breaking of these Si—C bonds, thus separating the corresponding organic species, e.g., organic radicals from the original silicone structure. While these radicals form a gas that disperses in the environment, the Si—O backbones of the now partially decomposed polymer remain as the sole part of the silicone that has not absorbed the UV photons. Meanwhile, each of the Si atoms in the polymer backbones are no longer fully interlinked except to two adjacent O atoms. This leaves two unpaired electrons per Si atom. Each of these electrons remains coupled to a corresponding positron in the atom nucleus and occupies a so-called orbital that is attached to the atom site. After laser irradiation of the original surface, these “dangling” bond electron orbitals constitute a dense one-dimensional network along each backbone on the actual silicone surface.

That network materializes the chemical “activation” of the processed silicone surface. In effect, and as a result of the laser-processing, the surface is no longer neutral, but is negatively charged. Eventually, an electric field is established that stems from these orbitals and tends to attract (i) positively charged species to form covalent bonding, or even (ii) neutral species that come to settle on the silicone surface and adhere to the Si—O backbones via electrostatic forces.

The end product of the laser-processed silicone surface is partially ablated and, therefore, engraved (i.e. recessed) down to some 10 μm or more below the original surface plane, depending on the number of super-imposed irradiations. The activated surface is, therefore, originally localized in the recessed area but is not limited to it, as explained by the discussion.

As noted above, C—H or other organic radicals are liberated during irradiation as free entities. The cloud of chemical species that is formed by these radicals tends to project outwards nanometer-scale particles (or nano-particles) of the silicone (Si—O) backbones. These nano-particles land on and populate the silicone surface area that is adjacent to the recessed laser-irradiated parts, thus contributing to the formation of a laser-activated silicone surface. Over that area, they form a dense layer of active species, since they contain those unpaired dangling bond electrons on each Si atom as mentioned above. Eventually, these species do react to the underlying virgin silicone surface, resulting in a strongly adherent, active cover. As a result, activation of the silicone surface is no longer restricted to the recessed laser-processed surface but extends eventually far beyond it.

This extended activation is conformal to the un-recessed, original silicone surface. The geometry of the conformal activated surface that surrounds the laser-recessed parts may be tailored through the actual geometry and distribution of these laser-processed recessed areas. Since the latter may be monitored by precisely positioning and/or scanning the laser beam onto the silicone surface, the entire conformal activated surface may be designed through computer-monitoring of the laser positioning on the silicone surface.

All silicones (including silicone rubbers) are accessible to the above-described laser-induced selective decomposition and activation. Such materials may differ by the type of organic-radicals that they contain. However, because each radical is connected to a single Si atom by a normal Si—C bond, different organic-radicals may be identically separated from their silicone backbone via identical irradiation conditions, irrespective of the individual identity of the organic-radicals and silicone formulation.

Three types of bonds are present in every silicone: Si—O, Si—C and C—H. The weakest of these bonds is Si—C (at 318 kJ/mol), the strongest is Si—O (at 452 kJ/mol), and C—H is intermediate in strength at 411 kJ/mol. Along with that bond hierarchy, optical absorption starts at 4.3, 5.3, and 5.5 eV, for Si—C, C—H, and Si—O bond (valence) electrons, respectively. Choosing a monochromatic beam working at 5 eV photon energy (i.e., 248 nm wavelength) restricts exclusively optical absorption to electrons belonging to Si—C bonds.

Increasing the actual power of a laser beam working at 5 eV should therefore allow the selective decomposition of silicone that preserves the original Si—O backbone and produces the formation of the dangling bond electrons that materialize the activation of the material. Comparatively, such 5 eV photons are not absorbed by silica additive parts. In contrast, they may be absorbed by crosslinker molecules, whether these are a silicone polymer or siloxane. In that case, again C—H and other organic radicals are selectively separated from the backbone of these molecules, without affecting their inter-linking function.

A suitable laser source that promotes this selective optical absorption to the most appropriate power is an excimer laser source working at 248 nm wavelength, i.e. 5.00 eV photon energy. Its actual instant power (i.e. beam energy/pulse duration) may vary in the range of 50 to 200 MW.

In one embodiment, the irradiation is pulsed (pulse duration being variable in the range 5 to 40 ns, full width, depending on manufacturer). Pulses are usually repeated several times along a train, at fixed time intervals. The processed material may be maintained fixed during irradiation, and the train of pulses processes the same area until a specific amount of ablated (activated) matter is produced. While being irradiated (i.e. during laser-scanning), the target polymeric material may also be displaced in front of the laser source on an X-Y table, moving perpendicularly to the laser beam axis. An appropriate combination of pulse repetition rate and scan velocity would ensure the required ablation per unit area. Material displacement can be computer-controlled to any geometry and scan-speed velocity.

The ablated species scatter around the laser-ablated area and establish the laser-activated silicone surface. Optionally, the extent of the scatter may either be limited to a few μm or expanded to several hundred μm, using a gas jet (e.g., an inert gas, such as He) that drifts the emitted species away from the irradiated area, and the scan geometry can be adapted to account for that scatter. In contrast, a monochromatic beam working at a photon energy exceeding 5.5 eV induces absorption from all valence electrons, irrespective of the bond type from which they originate. At and above an appropriate instant power level, this would eventually drive the full ablation of silicone with no activation of the remaining silicone surface, either of the irradiated part of it or of the surface area surrounding it.

Excimer lasers have been used to irradiate plastics to form metallized plastics. See U.S. Pat. No. 5,599,592 to L. Laude, entitled “Process For The Metallization of Plastic Materials and Products Thereto Obtained,” the entire contents of which are hereby incorporated by reference.

In an embodiment, a silicone patch having at least one activated surface formed by irradiation with laser light at a wavelength and power sufficient to eject organic species from the silicone article is prepared according to the method described above and further comprises one or more polypeptides comprising a disintegrin domain, as described below.

Opening the silicone backbone with an excimer laser (248 nm) and the resultant debris field created can effect the extent of binding by polypeptides comprising a disintegrin domain. In alternate embodiments, different laser patterns will achieve differing surface area opening of the silicone backbone as well as minimizing large debris. For example, different laser patterns can be employed such as in dots, lines and cross-hatching. Lased silicone can be assessed with SEM, AFM and profilometry to determine the surface roughness. Lasing can be adjusted to obtain a uniform layer of debris, leading to a uniform layer of polypeptides comprising a disintegrin domain. A more uniform surface can result in better sealing of the soft tissue injury by the patch.

In an embodiment, the time between lasing and protein application may affect the bonding uniformity and ultimately the attachment strength, as well as shelf life. Polypeptides containing a disintegrin domain can be applied 1 hour, 1 day, 3 days or 7 days after lasing. To assess binding uniformity of the polypeptides comprising a disintegrin domain, immunohistochemistry techniques and confocal microscope imaging can be used to determine how many binding sites are available in a planar slice of the patch at given height above the substrate. In addition, XPS (X-ray photoelectron spectroscopy) can be used on serial sections of the lased and disintegrin coated silicon. Standard silicone (and lased silicone without disintegrin) will yield only the characteristic XPS signals for oxygen (O), carbon (C) and silicon (Si). Following deposition and attachment of polypeptides containing a disintegrin domain to the activated surface, a nitrogen (N) peak is observed with XPS and the distance of the nitrogen from the surface can be determined. If this distance is <3 nm of the surface it is assumed the polypeptide is attached via a covalent linkage. Greater distances indicate weaker forms of binding.

Coupling of polypeptides containing a disintegrin domain to an activated silicone surface is generally restricted to the laser-activated areas as described above. When these structures are contained in a liquid solution, a drop of that solution may be disposed (e.g., manually) on the silicone surface. Only the parts of the surface that have been activated would retain the incoming species and ensure substantial adhesion and bonding. On non-activated surface areas, foreign species do not adhere to the virgin silicone surface and may, therefore, be removed by washing in water, gentle scrubbing, or tapping out without affecting those species that are strongly fixed on the activated silicone surface. Other means of disposing these foreign species may be practiced depending on the type and size of the species. For example, disposal may also be performed by evaporation in a vacuum chamber, and other physical or chemical means may be practiced as well without affecting the particular adhesion of these species to the laser-activated silicone surface alone.

Other polymers to which polypeptides comprising a disintegrin domain may be attached comprise polymers such as polyimide, polydimethylsiloxane, and parylenes, such as parylene N and C, and copolymer blends of silicone and non-silicone polymers. Non-silicones like the polyimides and parylenes, without being combined with a silicone based polymer, may not have activated surfaces when subjected to the excimer laser process, but are still useful for attaching to polypeptides comprising a disintegrin domain.

The patch can include other materials, such as to lend strength or permanence to the patch. The polymer component can be only part of the surface of the patch.

In an embodiment, the patch may be used in a number of tissue injuries, including in brain (e.g., cortex), heart, liver, and eye. “Soft tissue” includes organs, blood vessels, muscles, ligaments, tendons, cartilage, and nerves. As used herein, a “soft tissue injury” for the purposes of this application means cut, incision, avulsion, tear or puncture of a soft tissue. The injury can be a result of trauma or disease.

Different eye tissues display differential affinity to the patch's polymer surface linked to polypeptides comprising a disintegrin domain as disclosed herein. Highest affinity is observed for retina and conjunctiva. A moderate affinity is observed for cornea and sclera. Low affinity is observed for lens and choroid.

The patch can be directly applied to the target soft tissue using minimal pressure. The target soft tissue can be flushed of blood and exposed, either surgically or manually, and the polypeptide containing a disintegrin domain coated to the patch is pressed with minimal pressure onto the surface of the tissue. The binding to the tissue can occur within 10 seconds.

As used herein, “polypeptides comprising a disintegrin domain” refers to a class of amino acid sequences from cysteine-rich proteins that are potent soluble ligands of integrins and which are involved in regulating many processes such as cell-cell and cell-extracellular matrix adhesion, migration and invasion, cell cycle progression, differentiation and cell type specification during development of many metazoan organisms, and cell death and apoptosis. Polypeptides comprising a disintegrin domain are meant to include polypeptides derived from disintegrin proteins as obtained from snake venoms; polypeptides derived from disintegrin domains in mammalian ADAM proteins, including the 23 different disintegrin domains in the human family of ADAM proteins, and otherwise referred to herein as “AP” (“ADAM derived Polypeptide”); and uniquely designed polypeptides, designated MAPs (Modified ADAM-derived Polypeptides), a “modified” form of an AP, further described herein. The polypeptides may include one type of polypeptide (e.g. a single MAP) or may contain mixtures of polypeptides (e.g., multiple MAPs or CN plus VCN or CN plus one or more MAPs etc.)

The tri-peptide motif RGD (Arg-Gly-Asp) is conserved in many monomeric disintegrins and is located at the tip of a flexible loop, the integrin-binding loop, which is stabilized by disulfide bonds and protruding from the main body of the peptide chain. Many disintegrins purified from snake venoms bind to the fibrinogen receptor, integrin αIIbβ3, the binding of which results in the inhibition of fibrinogen-dependent platelet aggregation. Many disintegrins also bind to integrins αvβ3 (a vitronectin receptor) and α5β1 (a fibronectin receptor) in an RGD-dependent manner.

As used herein, “contortrostatin” (CN) refers to a polypeptides comprising a disintegrin domain isolated from Agkistrodon contortrix contortrix (southern copperhead) venom [2]. CN is produced in the snake venom gland as a multidomain precursor of 2027 bp having a 1449 bp open reading frame encoding the proprotein, metalloproteinase and disintegrin domains. The precursor is proteolytically processed, possibly autocatalytically, to generate mature CN. The full length CN preprotein is encoded by the nucleotide sequence 85-1536 of the full length mRNA (GeneBank AF212305), whereas the disintegrin domain of CN represents 1339-1533 of the mRNA. The CN disintegrin domain, which contains 65 amino acids, is shown below with the RGD sequence underlined.

(SEQ ID NO: 2)
DAPANPCCDAATCKLTTGSQCADGLCCDQCKFMKEGTVCRRARGDD
LDDYCNGISAGCPRNPFHA.

Mature CN includes two 65 amino acid polypeptides comprising a disintegrin domain linked together by 2 disulfide bridges. Based on structural data from other homodimeric polypeptides comprising a disintegrin domain [3], it is believed that the first and the third Cys residues of both 65 amino acid subunits pair to form two interchain disulfide bridges in an antiparallel fashion (the first Cys residue of one subunit pairs with the third one of the other subunit and vice versa).

CN displays the classical tripeptide RGD motif in its integrin-binding loop. Unlike other monomeric disintegrins from crotalid venoms, CN is a homodimer with a molecular mass (Mr) of 13,505 for the intact molecule and 6,750 for the reduced chains as shown by mass spectrometry [2].

As used herein, the term “purified” in reference to polypeptides (or proteins) does not require absolute purity. Instead, it represents an indication that the polypeptide(s) of interest is (are) in an environment in which the protein is more abundant (on a mass basis) than the environment from which the protein was initially produced. Purified polypeptides may be obtained by a number of methods including, for example, chromatography, preparative electrophoresis, centrifugation, precipitation, affinity purification, etc. The degree of purity is preferably at least 10%. One or more “substantially purified” polypeptides are at least 50% of the protein content of the environment, more preferably at least 75% of the protein content of the environment, and most preferably at least 95% of the protein content of the environment. Protein content may be determined using a modification of the method of Lowry et al. [4, 5], using bovine serum albumin as a protein standard.

Other useful polypeptides comprising a disintegrin domain include vicrostatin (VCN), as disclosed in U.S. patent Ser. No. 11/351,311, shown below:

GDAPANPCCDAATCKLTTGSQCADGLCCDQCKFMKEGTVCRRARGD
DLDDYCNGISAGCPRNPHKGPAT

The N-terminal G results from a post expression processing of an N-terminal thioredoxin fusion having a TEV protease linker site. The N-terminus of VCN may lack the G or may have some other amino acid(s) without impacting the activity of the molecule.

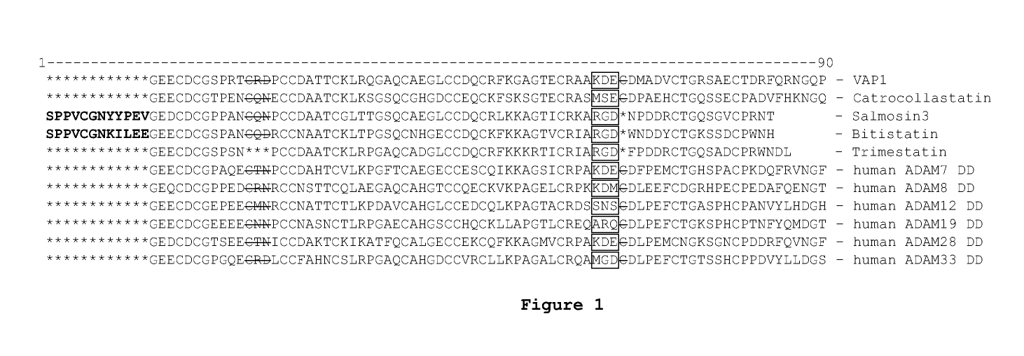

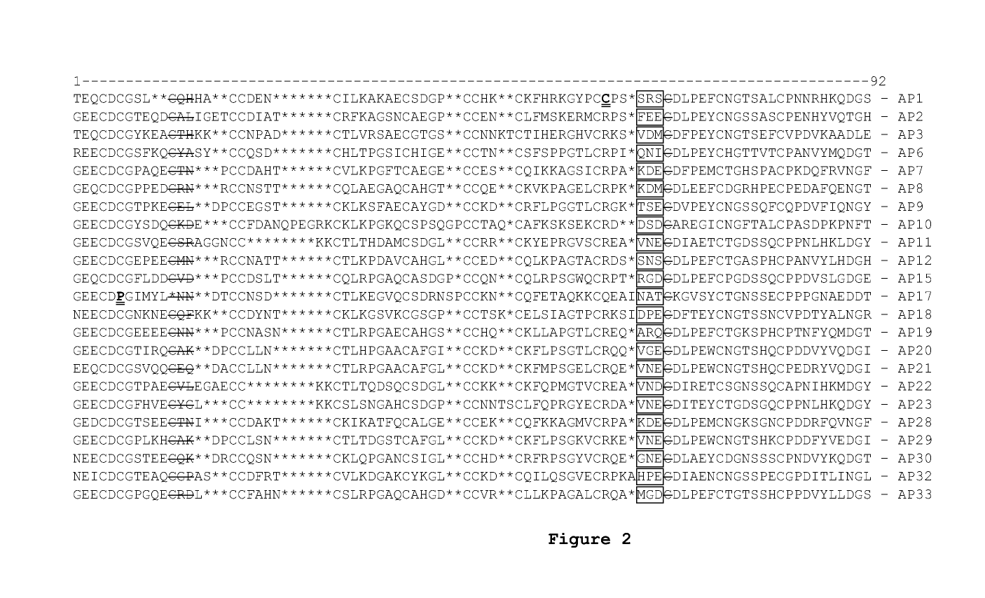

Other useful polypeptides comprising a disintegrin domain are a class of uniquely designed polypeptides, designated MAPs (Modified ADAM-derived Polypeptides), and encoding nucleic acids. As used herein, MAPs refer to a sequence modified form of the native disintegrin domain of an ADAM protein. As used herein, a “disintegrin domain of an ADAM protein” which may be referred to herein as “AP” (“ADAM derived Polypeptide”) is a disintegrin domain of the ADAM which has been separated from its metalloprotease and cysteine-rich domains and from any interdomain segments. Examples of APs are shown in FIG. 1 (from AP7-33) and in FIG. 2. The AP is a fragment of the ADAM but otherwise contains the native sequence of the corresponding segments of the ADAM. The N-terminal end of the AP is defined as the position 3 amino acid residues N-terminal from the CDC motif up to but not including the first cysteine N-terminal to the CDC. The C-terminal end of the AP is defined as the position 10 amino acid residues C-terminal from the 12th cysteine residue from the CDC motif up to but not including the next cysteine C-terminal to said 12^{th} cysteine residue. See e.g. FIGS. 1 and 2. There are two exceptions: (1) the C-terminal end of AP1 is defined as the position 10 amino acid residues C-terminal from the 13th cysteine residue from the CDC motif up to but not including the next cysteine C-terminal to said 13^{th} cysteine residue, and (2) ADAM17 has a CDP motif rather than a CDC motif from which the ends of the corresponding AP (AP17) are defined.

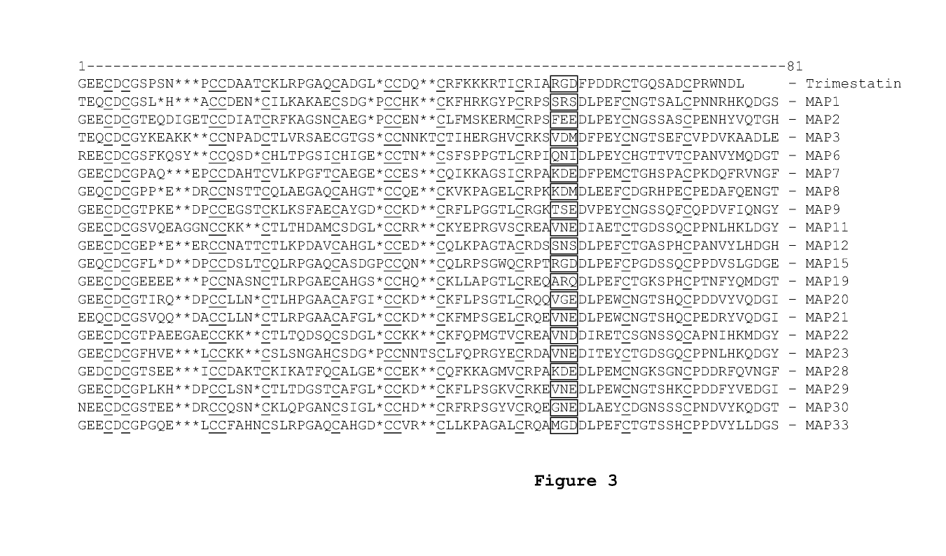

A “MAP” is a “modified” form of an AP, the modifications involving an alteration(s) in the sequence of the AP to achieve the beneficial properties described herein. MAPs, therefore, have sequences which are modified relative to the sequence normally present in the AP and corresponding sequence of the ADAM parent. As used herein, “modified” means that the amino acid is deleted, substituted or chemically treated and, in an embodiment, the alteration results in disruption of interdomain disulfide linkage. Exemplary MAPs are shown in FIG. 3. The MAP sequences are shown aligned with trimestatin, a prototypical medium-size snake venom disintegrin. All MAP constructs were modeled after medium-size snake venom disintegrins and had their sequences modified to fold similarly to these native snake venom molecules. The MAPs (except for MAP17) were constructed such that the first cysteine C-terminal to the CDC motif and two amino acids C-terminal to said cysteine as well as the cysteine C-terminal to the tripeptide motif of the corresponding AP is deleted. Alternatively, the cysteine residues can be substituted with alternate amino acids or the cysteine amino acid residues can be chemically modified such as to prevent disulfide bond formation. The amino acid substitutions can be conservative, e.g. the first cysteine C-terminal to the CDC motif of the AP can be substituted with a serine residue, the amino acid residues C-terminal to said cysteine can be substituted with a charged amino acid, or the cysteine C-terminal to the tripeptide motif can be substituted with a charged amino acid. Such mutational approaches and chemical treatments are well known in the art. With regard to chemical treatments, an example is the use alkylating agents to react with cysteine residues to prevent formation of disulfide bonds. Except for MAP10, 17, 18 and 32, MAPs display an 11 amino acid disintegrin loop, similar to the native loop of snake venom disintegrins. MAP 10 displays a 10 amino acid integrin loop and MAP17, MAP18, and MAP32 display a 12 amino acid disintegrin loop.

MAPs can be expressed and further purified as stand alone biologically active molecules in a bacterial system that supports both the generation of active soluble disulfide-rich polypeptides and high expression yields for these products. While not wishing to be held by theory, the MAPs were designed from the native APs so that they could adopt a snake venom disintegrin fold rather than their native ADAM conformations. The MAPs can be expressed with high yields in the Origami B (DE3) E. coli strain and further purified as stable and active free polypeptides that can interact with a class of mammalian cell surface receptors, the integrins, in a manner that is similar to that of native snake venom disintegrins. The MAPs also retain some of the signaling properties that are characteristic of the APs or disintegrin domain activities from the ADAM parent from which the MAP was derived form. For instance, retained characteristics may include signaling attributes related to the putative ability of the ADAM disintegrin domains to engage integrin receptors by utilizing amino acid residues located outside the classical disintegrin loop. Cellular functions of ADAMs are well known [6-11].

Although not wishing to be bound by theory, it is believed that the PII-class SVMPs that give rise to the prototypical medium-sized snake venom disintegrins (e.g., Trimestatin, Kistrin, Flavoridin etc) fail to form a critical disulfide bridge between the upstream spacer region and the disintegrin domain and thus the proteolytic attack happens in the residues located just N-terminally of where the disintegrin domain starts, the consequence of this being that the released medium-sized disintegrins are complete disintegrin domains containing no portion of the upstream spacer region. In contrast, it is believed that the PII-class SVMPs that give rise to the long-sized snake venom disintegrins (e.g., bitistatin, salmosin3 etc) fail to form a critical disulfide bridge between the metalloprotease domain and the downstream spacer region and consequently a proteolytic attack does happen more upstream in the spacer region with the release of a longer disintegrin.

Because in this case the proteolytic event is believed to happen upstream of a disulfide bridge that still forms between the spacer and the disintegrin domain, the long-sized snake venom disintegrins are released with a portion of the spacer region attached N-terminally to the freed disintegrin domain (see the sequence alignment of various disintegrin and disintegrin domains in FIGS. 1-3). Moreover, it is also believed that when the PII-SVMPs contain even more mutations and/or deletions, the disulfide bridges fail to form in the same spacer region but also in the N-terminal part of the disintegrin domain and even shorter variants of snake venom disintegrins are released (e.g., either partially truncated disintegrins domains that dimerize like contortrostatin or, more rarely, extremely truncated polypeptides like echistatin or eristostatin). It is further believed that in almost all cases, the free disintegrin domains display a conserved 11-amino acid disintegrin loop in the C-terminal half of their molecule, which is the hallmark of snake venom disintegrins.

The 23 different ADAM transcripts that have been identified in the human genome (3 of them being pseudogenes that are not normally translated into a protein product) have been modified as described herein to adopt the snake venom disintegrin fold.

Several ADAM transcripts have a number of isoforms. Nonetheless, inside the isoforms of different ADAMs the disintegrin domain's sequence is conserved and therefore there are only 23 different disintegrin domains in the human family of ADAM proteins. When produced recombinantly, the MAPs of the invention can interact in a high affinity manner with a defined integrin set. This property makes these mutant polypeptides broad spectrum integrin ligands for clinical and therapeutic use.

Similar to the other human ADAM members, the non-functional transcripts do contain complete disintegrin sequences that, if artificially translated in a recombinant system, can generate active polypeptides with novel biological functions. The disintegrin domains of human ADAMs have between 76 to 86 amino acids (the disintegrin domain of ADAM1 is the shortest, whereas that of ADAM10 is the longest), and with 2 exceptions (ADAMs 1 and 17), they all contain the 14 canonical cysteine residues of the original ADAM scaffold (see the aligned sequences of human ADAMs below). Unlike the snake venom disintegrins, that naturally evolved to function as platelet aggregation inhibitors, most which contain an RGD tripeptide motif at the tip of their disintegrin loop, the disintegrin loops of ADAMs display much different tripeptide motifs at their tips and therefore are expected to engage a broader range of integrins and in a different manner than their snake venom counterparts. In fact, each of the APs is believed to bind to a defined set of integrin receptors thus signaling in a unique manner (see FIG. 1 for the sequence alignment of ADAM and snake venom disintegrins illustrating the differences in the disintegrin loops).

The disintegrin domain of human ADAM15 contains a RGD tripeptide motif in its disintegrin loop which supports the hypothesis that human ADAM15 plays important regulatory roles in the cardiovascular system. This RGD tripeptide motif in ADAM 15 is shown as AP15 in FIG. 2.

MAPs for each AP portion of all 23 known human ADAM members were generated. The human ADAM disintegrin domain sequences were modified according to the rationale presented above, which includes removing the residues (among which include 2 cysteine residues) in the ADAM disintegrin domain that normally participate in interdomain-disintegrin domain disulfide bridge formation in the native ADAM proteins. Not wishing to be held by theory, the apparent function of these disulfide bridges is to keep the disintegrin loops in ADAMs tightly packed and unavailable to integrin receptors. By removing the residues that participate in the formation of these disulfide bridges, these MAPs acquire the mobility of the canonical 11-amino acid loop and the disintegrin-fold characteristic of snake venom disintegrins. Among the 23 members of the human ADAMs, 6 members perfectly fit the above-mentioned scheme (ADAMs 7, 8, 12, 19, 28 and 33) when aligned with long- and medium-sized snake venom disintegrins as well as with PIII-class SVMPs (see FIG. 1 for an alignment of snake venom disintegrins and human ADAM disintegrin domains). Nonetheless, by introducing these modifications, with the exception of 4 ADAMs (10, 17, 18 and 32), all human ADAM members were converted to MAPs that display a 11-amino acid disintegrin loop. Regarding the 4 exceptions, 3 (ADAMs 17, 18 and 32) were converted to MAPs displaying a slightly longer, 12-amino acid loop, while 1 member (ADAM 10) was converted to a MAP carrying a slightly shorter 10-amino acid disintegrin loop (see AP10 in FIG. 2 for a sequence alignment). Moreover, in the case of 2 APs (ADAMs 1 and 17), one additional native residue in each sequence was replaced with either an arginine residue (to generate MAP1) or a cysteine residue (to generate MAP17) to restore the cysteine pattern characteristic of disintegrin domains (see FIG. 2 for sequence alignment).

As used herein, “interdomain regions” or “spacer regions” means the polypeptide portion of an ADAM between the metalloprotease and disintegrin domain (the “MD interdomain region”) and between the disintegrin domain and the cysteine-rich domain (the “DC interdomain region”), respectively, wherein the MD interdomain region starts at least 10 amino acid residues N-terminal to the AP and the DC interdomain region starts at least 10 amino acid residues C-terminal to the AP. Each interdomain is 5 to 15 amino acids in length.

The DNA sequences of all 23 MAPs were de novo synthesized and cloned into the pET32a expression vector [30] downstream of bacterial thioredoxin A (TrxA). The MAPs were produced in the Origami B (DE3) bacterial strain as described in PCT Patent Application No. PCT/US09/64256, filed Nov. 12, 2009, and titled “Method of expressing proteins with disulfide bridges with enhanced yields and activity.” This application describes an improvement upon the expression system disclosed in U.S. Publication no. 20060246541 which includes, as an embodiment, expression of a chimeric snake venom disintegrin vicrostatin (VCN) in the Origami B (DE3)/pET32a system. The improved method was used to generate increased amounts of correctly-folded active MAPs. This is achieved by growing the Origami B cells in a less selective environment and thus allowing for the generation and expansion of VCN-transformants that display a more optimal redox environment during the induction of the heterologous recombinant protein production. Unlike other E. coli strains, the Origami B is unique in that, by carrying mutations in two key genes, thioredoxin reductase (trxB) and glutathione reductase (gor), that are critically involved in the control of the two major oxido-reductive pathways in E. coli, this bacterium cytoplasmic microenvironment is artificially shifted to a more oxidative redox state, which is the catalyst state for disulfide bridge formation in proteins [12, 13].

The Origami B strain has growth rates and biomass yields similar to those obtained with wild-type E. coli strains, which makes it an attractive and scalable production alternative for difficult-to-express recombinant proteins like VCN. This strain is also derived from a lacZY mutant of BL21. The lacY1 deletion mutants of BL21 (the original Tuner strains) enable adjustable levels of protein expression by all cells in culture. The lac permease (lacY1) mutation allows uniform entry of IPTG (a lactose derivative) into all cells in the population, which produces a controlled, more homogenous induction. By adjusting the concentration of IPTG, the expression of target proteins can be optimized and theoretically maximal levels could be achieved at significantly lower levels of IPTG. Thus the Origami B combines the desirable characteristics of BL21 (deficient in ompT and lon proteases), Tuner (lacZY mutant) and Origami (trxB/gor mutant) hosts in one strain. As mentioned above, the mutations in both the thioredoxin reductase (trxB) and glutathione reductase (gor) greatly promote disulfide bond formation in the cytoplasm [13].

Although the Origami B strain offers a clear advantage over E. coli strains with reducing cytoplasmic environments like BL21 (FIGS. 1 and 2 show a comparison in expression levels between strains), the mere usage of the Origami B strain and the pET32a expression vector does not automatically guarantee the generation of a soluble and/or active product. The generation of disulfide-rich polypeptides in Origami B appears to be sequence dependent. For example, MAPs (e.g. MAP9 and MAP15) can be expressed in Origami B with significantly higher expression yields compared to their corresponding AP versions of human ADAMs 9 and 15 despite the fact that the same system and production technique were employed (FIGS. 1 and 2). Consequently, the modification of APs into MAPs can result in polypeptides having a disintegrin domain with greater expression yield in Origami B cells.

Furthermore, after purifying expressed disintegrin domains (APs) of ADAM 9 and 15, in a process that involves TEV protease treatment and RP-HPLC purification, the collected free polypeptides appeared to be unstable and to precipitate out of solution after reconstitution from lyophilized powder. In contrast, the corresponding MAP polypeptides, generated by employing the same purification steps, appear to be much more soluble and stable when reconstituted in water after lyophilization.

Polypeptides comprising a disintegrin domain are prepared as described herein so as to be substantially isolated or substantially purified. As used herein, the term “substantially purified” (or isolated) in reference to polypeptides comprising a disintegrin domain does not require absolute purity. Instead, it represents an indication that a preparation of polypeptides comprising a disintegrin domain are preferably greater than 50% pure, more preferably at least 75% pure, and most preferably at least 95% pure, at least 99% pure and most preferably 100% pure. Polypeptides comprising a disintegrin domain can be prepared synthetically or prepared by recombinant expression.

The term “substantially” as used herein means plus or minus 10% unless otherwise indicated.

Compositions of the invention may also be patches coated with drugs to deliver the drug to the attachment area and, for example, speed healing of the injury site. Other uses are possible and drugs that can be used in such approaches include, for example, chemotherapeutics and antibiotics.

Compositions of the invention may also be patches that can provide immediate, reliable, and reversible fixation to the CNS for use in neurostimulation, drug delivery, and implantable sensors. Clinical applications include therapeutic rehabilitation of a number of neurological diseases including brain and spinal cord trauma. In an embodiment, compositions of the invention can be scaffolds that guide axon regrowth (after trauma) for attachment to remaining neural structures.

To remove soft tissue patches from soft tissue after attachment, one can apply an enzyme to cleave the integrin(s) exposed on the surface of cells, thereby breaking the bond between the disintegrin-linked polymer surface of the patch and adjacent integrins. Such enzymes can include, but are not limited to, plasmin, trypsin, pepsin, collagenase, elastase, endoproteinase Glu C, endoproteinase Asp N and Factor Xa protease.

In an embodiment, the polymer surface of the patch linked to one or more polypeptides comprising a disintegrin domain is used as a substrate to support the growth of tissue culture cells. The cultured cells can be stem cells. The stem cells can be embryonic retinal progenitor cells. In addition cancer cells (prostate, breast, ovarian, melanoma, glioma, and cancer cells from other solid tumors) as well as endothelial cells can be grown using the patch coated with one or more polypeptides comprising a disintegrin domain.

### EXAMPLES

### Example 1

### Protein Attachment to a Silicone Surface

Snake venom disintegrin (contortrostatin) is a homodimeric polypeptide that contains an RGD amino acid segment and disulfide bonds that allow the protein to attach to activated silicone. An excimer laser was used to physically break the molecular bonds and produce dangling free bonds on the silicone surface. Using a pipette, the contortrostatin was dropped onto the lased silicone surface and allowed to dry.

### Example 2

### Preparation of Retinal Tissues

Postmortem porcine eyes were prepared by removing the vitreous humor with a vitreous cutter (Bausch and Lomb). The posterior segment of the eye was flattened by making four cuts in four different quadrants from the pars plana to the equator. The eye was pinned out onto a polystyrene surface and quadrants of the retina were delicately removed. Each piece of retina was glued (Adhesive Systems RP 1500 USP) face up (i.e. internal limiting membrane up) to a piece of aluminum and allowed to dry for 10 minutes. During this time the retina was kept moist with drops of saline.

### Example 3

### Protein Adhesive Strength

The adherence forces between the contortrostatin-coated silicone and the retina were measured by dynamic mechanical analysis, using a Bose ElectroForce 3100. Contortrostatin-coated silicone was glued (Adhesive Systems RP 1500 USP) to a piece of plastic and lowered onto the prepared retina. The silicone piece was raised 4 mm over 10 seconds and the adhesive forces resulting from the separation of retina and aluminum were recorded.

After the excimer laser was used to physically break molecular bonds, the contortrostatin drop can be seen absorbing into the lased areas and later extending over the silicone debris on the surface. To test the adhesive strength of the protein to the silicone, a simple scotch tape test was performed. The scotch tape could not be removed from the activated surface.

### Example 4

### Adhesive Strength to the Retina

Dynamic mechanical analysis of contortrostatin-coated silicone and non-laser processed silicone was performed. The silicone in each case was removed from the retina at 0.4 mm/second. The adhesive force of the contortrostatin-coated silicone is approximately 340 mN, at which point the retina was torn away from the aluminum surface whereas the plain (non-activated) silicone is easily detached from retina after just 10 mN.

### Example 5

### Full Thickness Eyewall Tear Model in Dogs

A study dog is anesthetized under standard protocols. The eye to be tested is cleaned and draped under aseptic precautions. The eye lids are kept apart with an eyelid speculum. A one cm full thickness eyewall tear (conjunctiva, sclera, choroid and retina) is created with a MVR (microvitreoretinal) blade. A patch is applied over the tear on the conjuctiva (or other injured eye structure) and antibiotic eyedrops applied. The dog is examined every three days to study the intraocular pressure and healing of the eyewall tear. The patch is enzymatically removed 2 weeks after attachment and the eyewall is repaired permanently.

### Example 6

### Retinal Tear Model

The animal is anesthetized under standard protocols. The eye to be tested is cleaned and draped under aseptic precautions. The eye lids are kept apart with an eyelid speculum. A three-port vitrectomy opening is made in the pars plana. An infusion cannula is placed in the inferior temporal quadrant sclerotomy and fixed with sutures. Two superior sclerotomies are made in the superior temporal and superior nasal quadrants. With infusion from the infusion cannula, a partial vitrectomy is performed. Following this step, a 1 cm full thickness retinal tear is created with a MVR (microvitreoretinal) blade. To simulate a trauma scenario, the tear is repaired in a second surgery, one week later. The surgical preparation is as described above (three-port vitrectomy). The edges of the retinal tear are brought together with a silicone patch. The animals are followed for up to 6 months to observe the condition of the retina and status (attached or not) of the patch.

### Example 7

### Retinal Patch Surgery

With the animal under anesthesia, the eye is prepped for a pars plana approach, and the pupils dilated using 10% phenylephrine hydrochloride and 1% tropicamide eye drops. A lateral canthotomy followed by a 360-degree conjunctival peritomy is performed and the rectus muscles isolated. The intended position of implant is determined. A sclerotomy is made posterior to the limbus in the inferior temporal quadrant in order to place an infusion of BSS, and infusion started. Using these sclerotomies, a vitrectomy is performed using techniques commonly used to strip the posterior hyaloid away from the retina. After the vitrectomy, the superotemporal port is enlarged to allow introduction of the patch into the eye, with intravitreal end-gripping forceps (Grieshaber Co., Inc. Kennesaw, Ga.) until it is attached to the retina. During this step in the procedure, the intraocular pressure is raised by adjusting the height of the infusion fluid bottle to prevent any choroidal bleeding. The sclerotomies are closed and the conjunctiva is pulled over the device and reattached at the limbus. The lateral canthotomy area is sutured.

### Example 8

### Implant Evaluation Protocols

Optical coherence tomography (OCT) is an imaging method that uses reflected light to provide information about retinal structure. The resolution ranges from 2 to 10 μm, depending on the system. This helps in not only understanding the interaction between the device and the tissue, but also helps to study the effects of chronic implantation on the tissue beneath. The canines undergo a baseline evaluation OCT, followed by a monthly evaluation. The canines are anesthetized and the pupils dilated. Fundus photography is used to document the progression of any gross anatomical changes or the absence thereof in the implanted eyes. Fluorescein angiography is a diagnostic test that allows study of the retinal blood flow, which helps to determine if there is any acute or chronic damage to the retinal or choroidal blood flow due to the device presence in the eye. Both eyes are dilated after a routinely accepted anesthetic procedure and pictures taken from the front and the back of the eye with a fundus camera. Fluorescein dye is then injected through a large vein and pictures taken sequentially to document the progress of the dye as it flows through the retinal and choroidal vessels. This procedure is done once every month.

Electrophysiological techniques are used to evaluate the excitability and viability of the retina to light and electricity. Electroretinography (ERG) is used to record retinal activity to light stimuli.

Once the animal completes the study duration, or is documented to develop an adverse event like endophthalmitis, the animal is euthanized. Pre-approved procedures are undertaken to remove the eyes for gross dissection and micro-histopathology. Both eyes are enucleated, and fixed in 4% paraformaldehyde. Gross dissection are undertaken to document the changes, if any, in the eye due to the chronic implantation of the device. Once the gross dissection is complete, the eye is sectioned to make microscopic slides for study. Five-μm sections are made on the microtome, and two of these sections are placed on one slide and stained for the study.

### Example 9

### Preparation of Maps

Bacterial cells and reagents. The Origami B (DE3) E. coli strain and pET32a expression vector carrying the bacterial thioredoxin A gene (trxA) were purchased from Novagen (San Diego, Calif.). All 23 MAP DNA sequences were de novo synthesized and delivered into a plasmid by Epoch Biolabs, Inc. (Sugar Land, Tex.). The AP DNA sequences were PCR amplified from cDNA libraries built from several mammalian cell lines including HUVEC (PromoCell GmbH, Heidelberg, Germany), MDA-MB-435 (ATCC, Manassas, Va.), MDA-MB-231 (ATCC, Manassas, Va.), and Jurkat (ATCC, Manassas, Va.). The oligonucleotide primers used for further cloning the APs and MAPs DNA sequences into pET32a expression vector were synthesized by Operon Biotechnologies, Inc. (Huntsville, Ala.). All restriction enzymes and ligases used for cloning the APs and MAPs DNA sequences into pET32a expression vector were purchased from New England Biolabs, Inc. (Ipswich, Mass.). The recombinant TEV protease was purchased from Invitrogen (Carlsbad, Calif.).

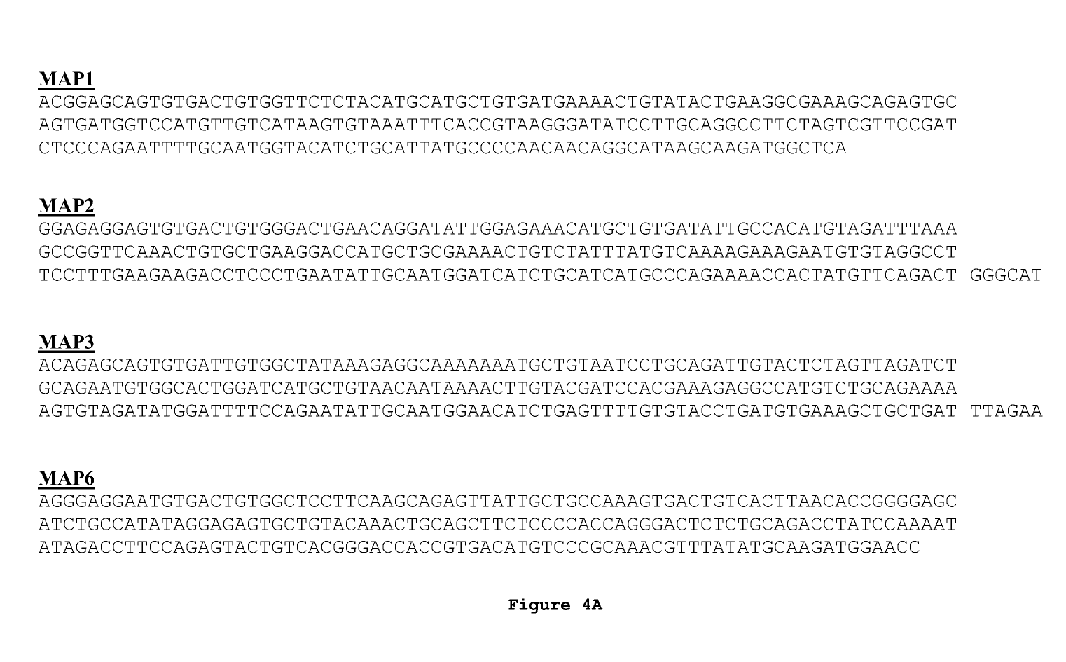

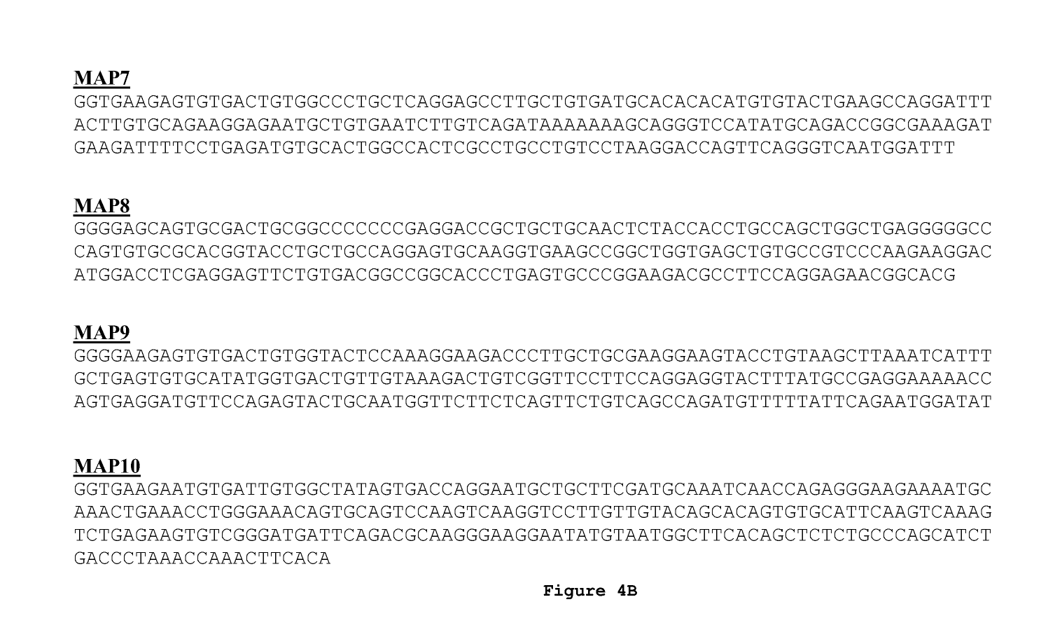

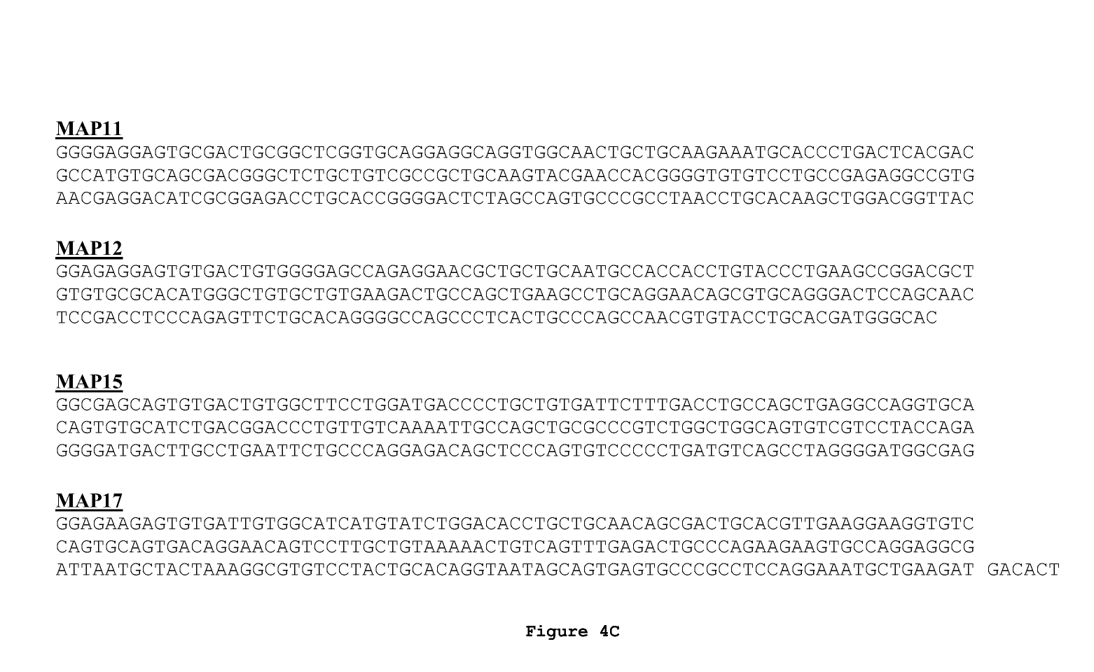

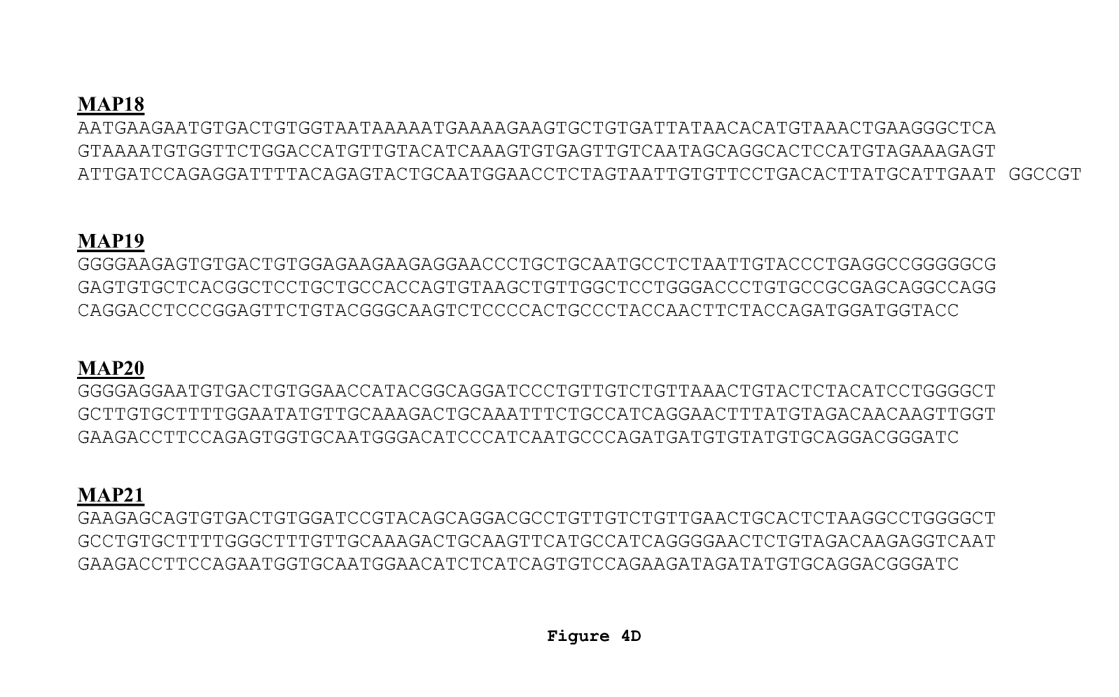

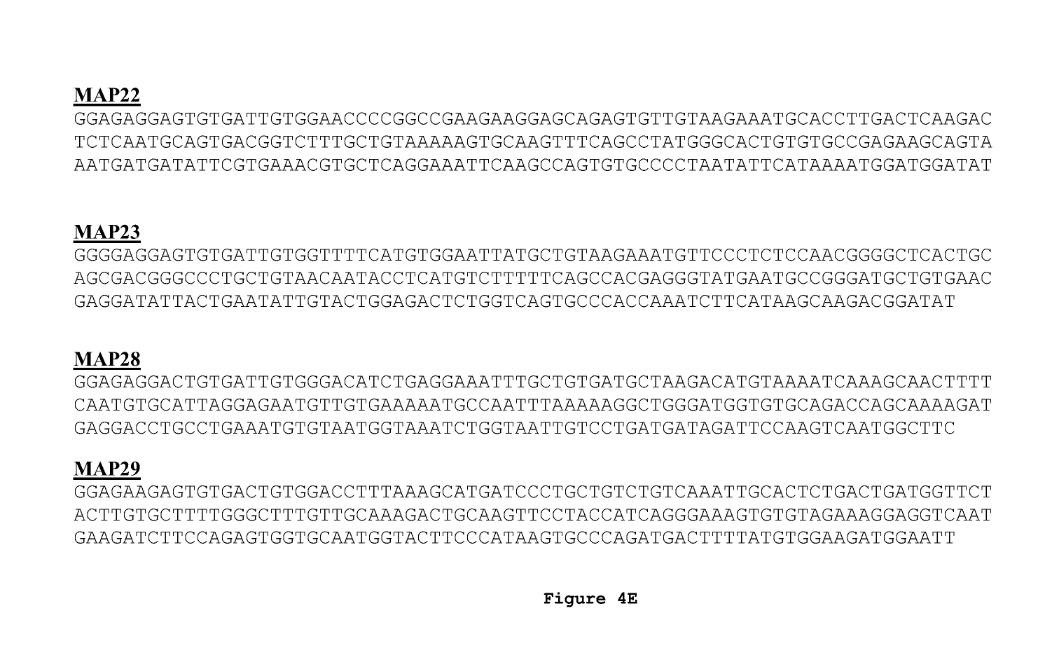

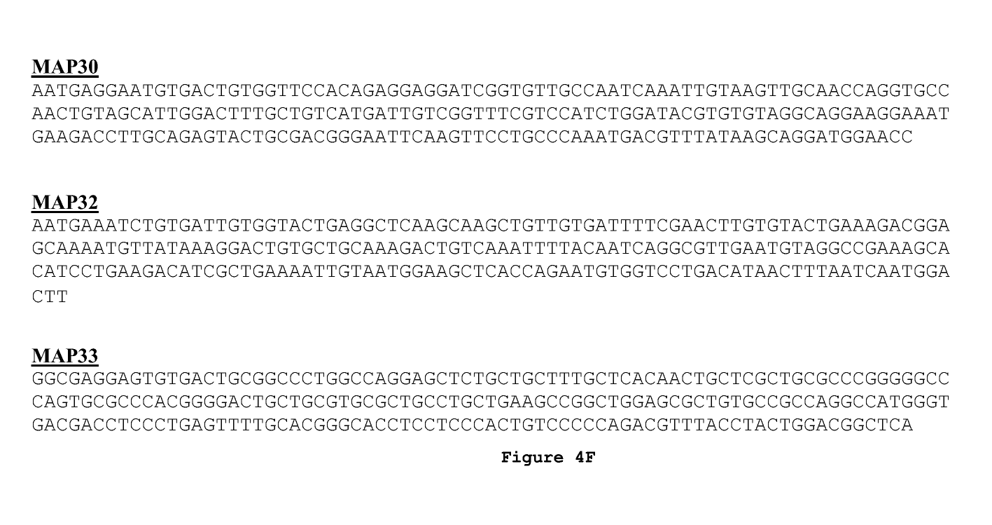

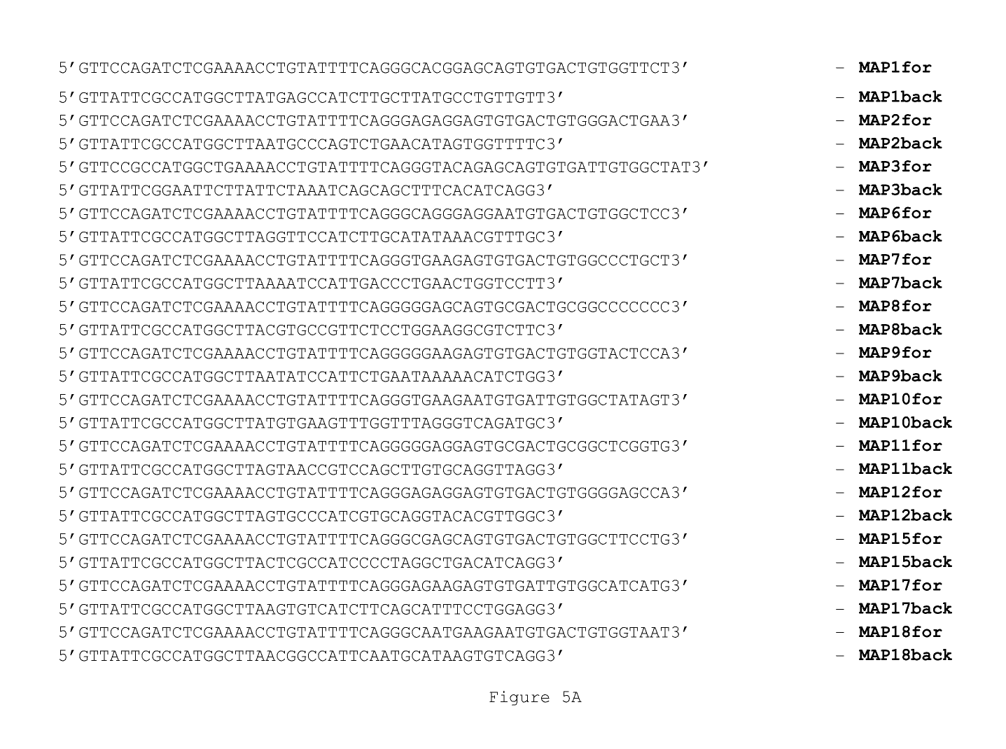

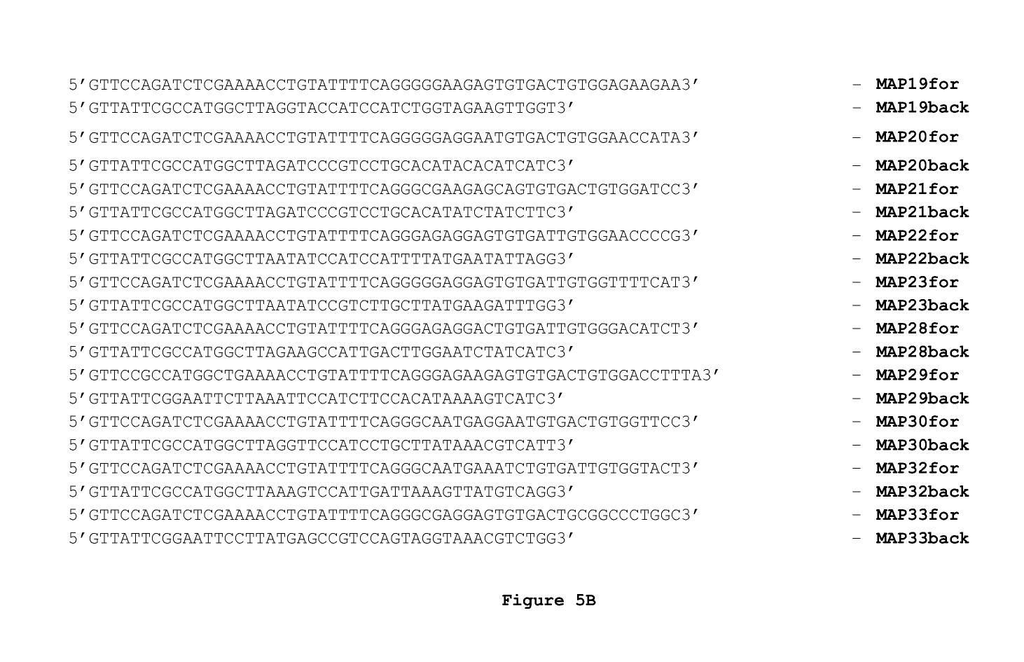

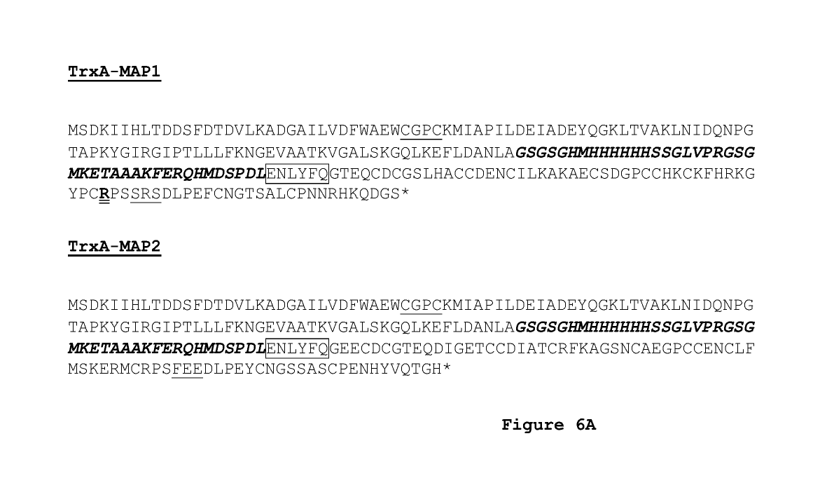

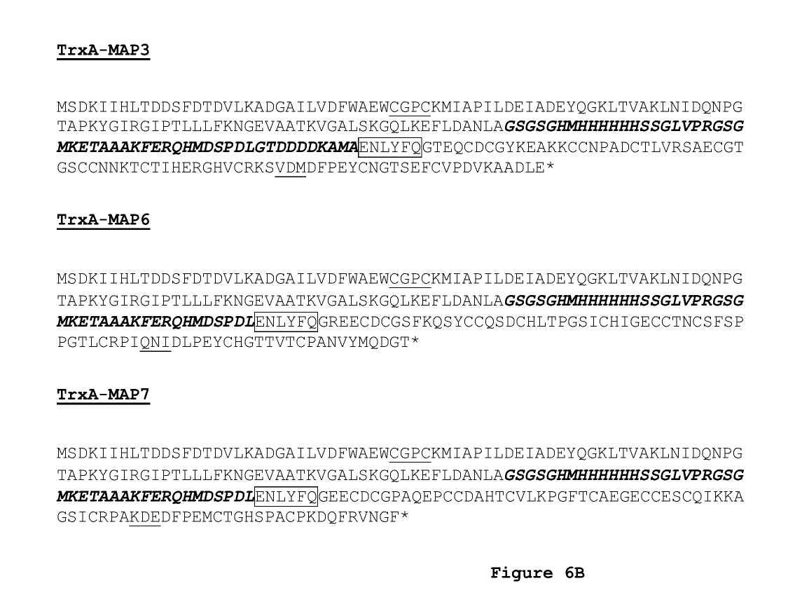

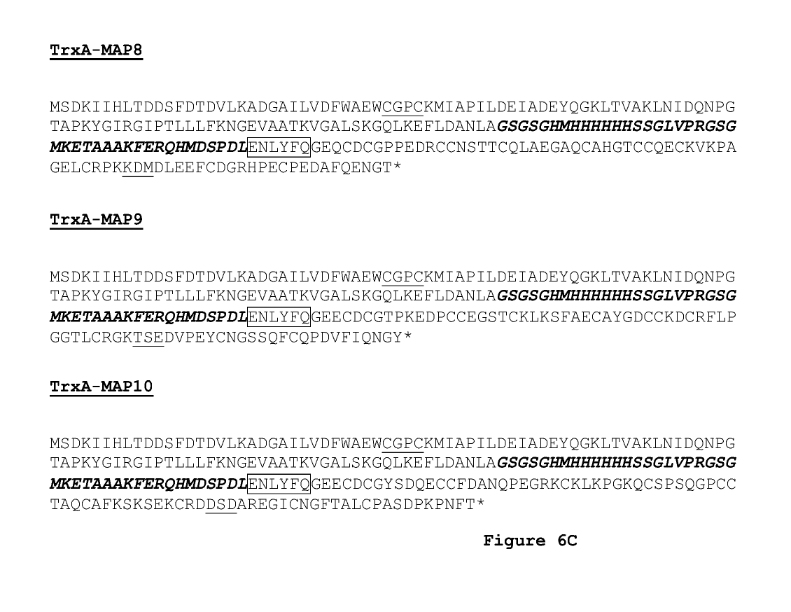

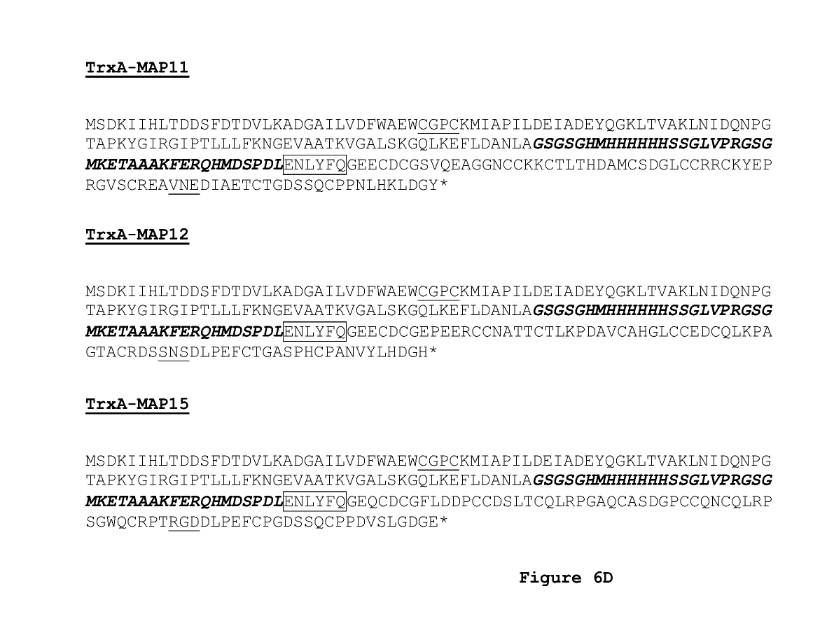

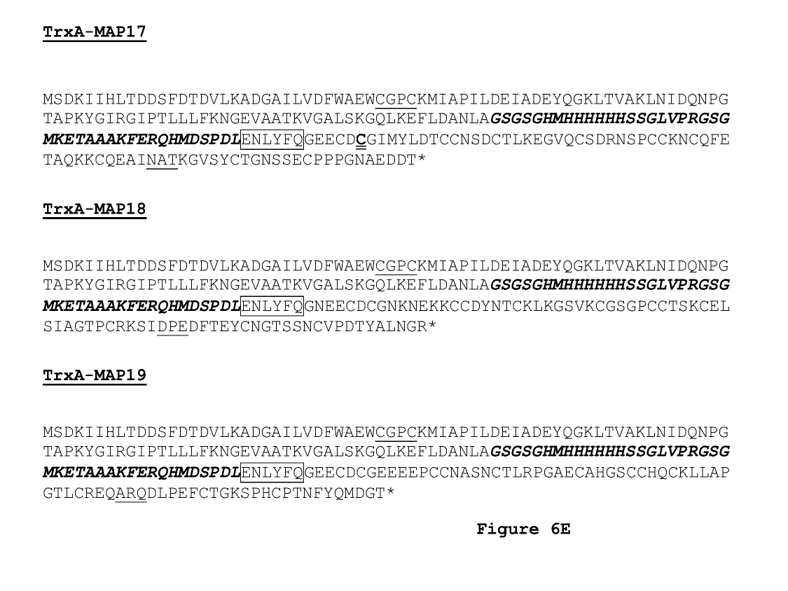

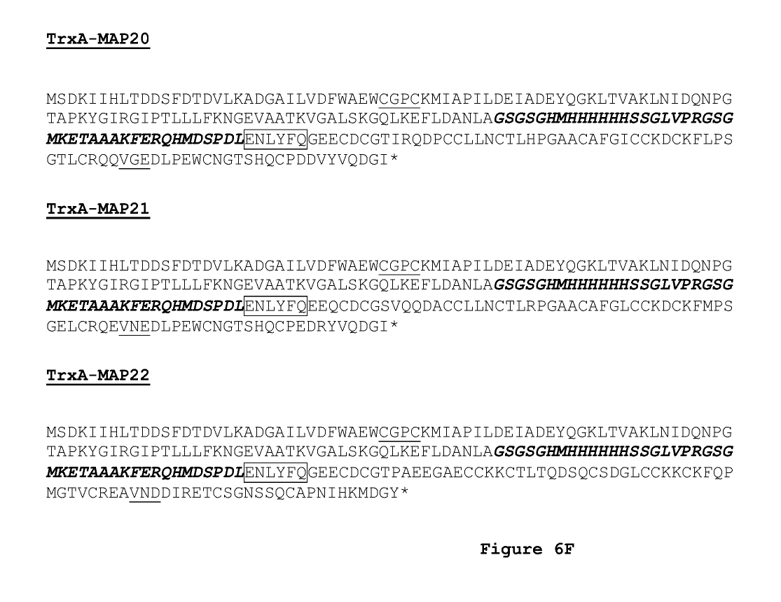

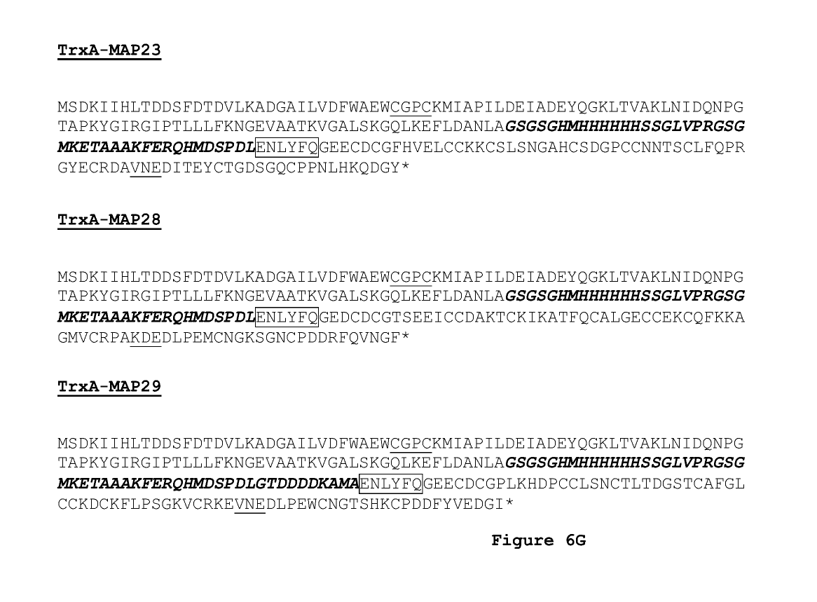

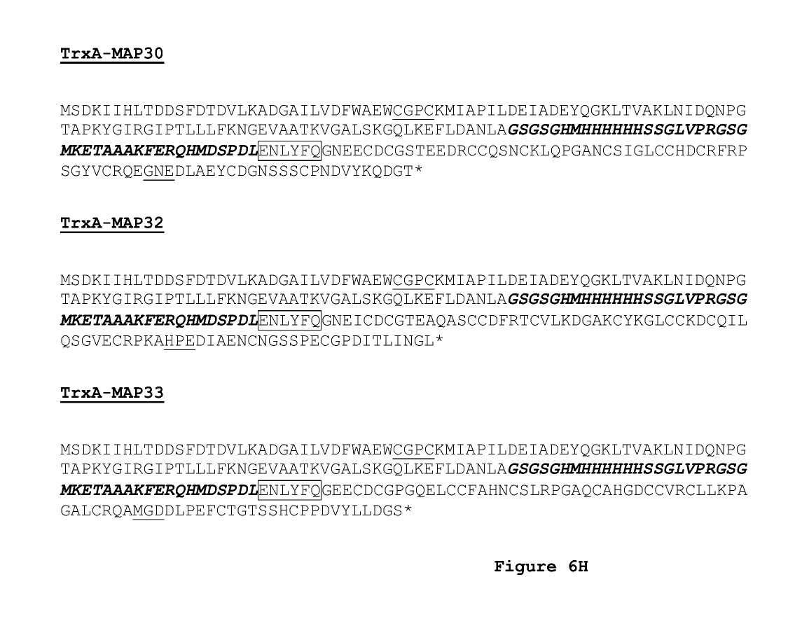

Construction of MAP expression vectors and recombinant production. The synthetic MAPs DNA sequences that were cloned into pET32a expression vector downstream of TrxA are listed in FIG. 4A-F. The oligonucleotide primers used for MAPs cloning are listed in FIG. 5A-B. The generated pET32a plasmids carrying the DNA sequences of MAPs cloned downstream of TrxA gene were initially amplified in DH5α E. coli, purified and sequenced before being transferred into Origami B (DE3) E. coli. The amino acid sequences of the fusion proteins are shown in FIG. 6A-H. The transformed cells for each MAP construct were then plated on LB-Agar supplemented with carbenicillin (50 μg/mL), tetracycline (12.5 μg/mL), and kanamycin (15 μg/mL) and grown overnight at 37° C. From these plates, multiple cultures were established for each MAP construct from individual colonies of transformed Origami B by transferring these colonies into LB media containing carbenicillin (50 μg/mL). These initial cultures were grown overnight and further used for the inoculation of bigger volumes of LB media containing carbenicillin (50 μg/mL) that were grown at 37° C. and 250 rpm in a shaker-incubator until they reached an OD600 of 0.6-1. At this point, the cells from individual MAP cultures were induced using 1 mM IPTG and incubated for another 4-5 hours at 37° C. and 250 rpm. At the end of the induction period, the cells from individual MAP cultures were pelleted at 4000×g and lysed in a microfluidizer (Microfluidics M-110L, Microfluidics, Newton, Mass.). The operating conditions of the microfluidizer included applied pressures of 14,000-18,000 psi, bacterial slurry flow rates of 300-400 ml per minute and multiple passes of the slurry through the processor. The insoluble cellular debris from lysates processed from individual MAP cultures was removed by centrifugation (40,000×g) and the soluble material containing Trx-MAPs for each MAP culture was collected. The expressed fusion proteins (i.e., Trx-MAPs) in the collected soluble lysates were then proteolysed by incubation with recombinant TEV protease overnight at room temperature which efficiently cleaved off each individual MAP from its TrxA fusion partner as monitored by SDS-PAGE. When proteolysis was complete, the proteolyzed lysates were passed through a 0.22 μm filter, diluted 1:100 in double distilled H₂O, ultrafiltrated through a 50,000 MWCO cartridge (Biomax50, Millipore) and then reconcentrated against a 5,000 MWCO cartridge (Biomax5, Millipore) using a tangential flow ultrafiltration device (Labscale TFF system, Millipore).

The APs were cloned into pET32a, transformed into Origami B, and expressed using the same procedures described above for MAPs.

Purification of recombinant MAPs. The MAPs were purified from filtrated lysates by employing a high-performance liquid chromatography (HPLC) procedure according to a protocol previously established for snake venom disintegrins [2]. Purification was performed by C18-reverse phase HPLC using the standard elution conditions previously employed for the purification of native CN [2]. Individual filtrated lysates processed as described above were loaded onto a Vydac C18 column (218TP54, Temecula, Calif.). A ten-minute rinse (at 5 ml/min) of the column with an aqueous solution containing 0.1% TFA was followed by a linear gradient (0-100%) elution over 150 min in a mobile phase containing 80% acetonitrile and 0.1% TFA. The MAPs start eluting in 35-40% acetonitrile.

High yields were obtained for MAPs. For example, both MAP9 and MAP15 were generated in Origami B (DE3) with batch-to-batch expression yields ranging from 200 mg to 350 mg of HPLC-purified protein per liter of bacterial culture. These high yields of purified recombinant MAPs were achieved by lysis of the pelleted bacterial transformants at the end of the induction step with a microfluidizer. In general, higher resulting yields were observed for the purification of MAPs as compared to the corresponding APs.

### Example 10

### Adherence of CN or VCN-Coated Activated Silicone to Different Structures of the Eye

We examined the ability of the VCN or CN-silicone patch to bind to porcine ocular tissues: conjuctive, retina, cornea, sclera, lens and choroid. For each ocular tissue type, a section of tissue was placed at the bottom of a saline filled vessel and a VCN or CN-silicone attached to a screw head was lowered onto the tissue at 37° C. in vitro. The binding strength as a function of presence or absence of blood at concentrations of 10, 25, 50 and 100 ml/L was compared. Quantitative measures were used to assess adherence.

Plain silicone and lased silicone controls did not stick to any tissues. CN and VCN-coated activated silicone strongly adhered to conjunctiva and retina. CN and VCN-coated activated silicone moderately adhered to cornea and sclera but did not adhere to lens or choroid.

### Example 11

### Adherence of CN-Coated Activated Silicone to Liver

A similar assay to the retinal assay was employed, except liver tissue was used.

The ability of CN-silicone to adhere to other tissues was investigated using binding to liver CN-silicone attached to a screw head. The activated silicone-covered screw is lowered onto the surface of a section of rabbit liver at the bottom of a saline chamber (37° C.) and tested for its ability to raise the piece of liver off the chamber bottom when the activated silicone is uncoated and when the activated silicone is coated with CN. The CN-silicone screw was able to lift the piece of liver off the chamber bottom, whereas the uncoated silicone screw was not.

Removing the liver from the CN-silicone screw tears the tissue with a portion remaining attached to the screw, thereby indicating strong binding between CN-silicone and other tissue such as liver.

### Example 12

### Cultured Growth of Rat Embryonic Retinal Progenitor Neurons

Mammalian cells grown in culture display optimal growth and proliferation profiles when grown on tissue culture treated plastic (a rough positively charged surface) or on plasticware coated with ECM proteins. Growing on ECM proteins is a close approximation of the type of environment and contacts cells would experience in a living organism. The presence of the ECM proteins, which contain attachment motifs for integrins on the cell surfaces, allows the cells to adhere and proliferate on a surface. Disintegrins display similar integrin binding motifs and have been shown to be able to support cellular attachment and growth. To determine if the CN-silicone was capable of supporting cell growth and proliferation, rat embryonic retinal progenitor neurons (cultured from sacrificed rat tissues) were plated and grown on lased silicone both with and without CN in complete media. We were able to demonstrate the effect the disintegrin coating has on cell growth and proliferation. Microscopic observation showed that without the disintegrin coating the cells clump, do not spread, and are loosely attached. By contrast, the presence of the disintegrin provides a surface the cells are able to bind to allowing for the characteristic spread of the cells into a confluent monolayer.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

### CITED REFERENCES

1. Zhou, Q, et al., Molecular cloning and functional expression of contortrostatin, a homodimeric disintegrin from southern copperhead snake venom., Arch Biochem Biophys 2000. 375(2): 278-88.
2. Trikha, M., et al., Purification and characterization of platelet aggregation inhibitors from snake venoms. Thromb Res, 1994. 73(1): 39-52.
3. Bilgrami, S., et al., Crystal structure of schistatin, a disintegrin homodimer from saw-scaled viper (Echis carinatus) at 2.5 A resolution. J Mol Biol, 2004. 341(3): 829-37.
4. Lowry, O. H., et al., Protein measurement with the Folin phenol reagent. J Biol. Chem., 1951. November; 193(1):265-75.
5. Hartree E. F., Determination of protein: a modification of the Lowry method that gives a linear photometric response. Anal Biochem., 1972 August; 48(2):422-7.
6. Edwards, D. R., M. M. Handsley, and C. J. Pennington, The ADAM metalloproteinases. Mol Aspects Med, 2008. 29(5): p. 258-89.
7. Mochizuki, S, and Y. Okada, ADAMs in cancer cell proliferation and progression. Cancer Sci, 2007. 98(5): p. 621-8.
8. Reiss, K., A. Ludwig, and P. Saftig, Breaking up the tie: disintegrin-like metalloproteinases as regulators of cell migration in inflammation and invasion. Pharmacol Ther, 2006. 111(3): p. 985-1006.
9. Tousseyn, T., et al., (Make) stick and cut loose—disintegrin metalloproteases in development and disease. Birth Defects Res C Embryo Today, 2006. 78(1): p. 24-46.
10. Arribas, J., J. J. Bech-Serra, and B. Santiago-Josefat, ADAMs, cell migration and cancer. Cancer Metastasis Rev, 2006. 25(1): p. 57-68.
11. Duffy M. J. et al., Role of ADAMs in cancer formation and progression. Clin Cancer Res. 2009 Feb. 15; 15(4):1140-4.
12. Bessette et al., Efficient folding of proteins with multiple disulfide bonds in the Escherichia coli cytoplasm. Proc. Natl. Acad. Sci. (USA) 1999, 96(24):13703-8.
13. Prinz, W. A., et al., The role of the thioredoxin and glutaredoxin pathways in reducing protein disulfide bonds in the Escherichia coli cytoplasm. J Biol Chem, 1997. 272(25): p. 15661-7.

That which is claimed is:

1. A method for treating a soft tissue injury in an individual comprising applying to the surface of said soft tissue injury a patch comprising a polymer, said polymer being present on a surface of said patch, said polymer of said surface linked to polypeptides comprising a disintegrin domain wherein said polypeptides facilitate attachment of the patch to the site of the soft tissue injury, thereby treating said soft tissue injury.

2. The method of claim 1 wherein said polymer comprises silicone and at least part of said silicone of said surface is activated.

3. The method of claim 2 wherein said silicone of said surface is activated by irradiation with laser light at a wavelength and power sufficient to eject organic species from the silicone substrate.

4. The method of claim 1 wherein said polymer comprises a parylene.

5. The method of claim 1 wherein said polypeptides comprising a disintegrin domain is contortrostatin (CN) or vicrostatin (VCN).

6. The method of claim 1 wherein said polypeptides comprising a disintegrin domain is an ADAM-derived polypeptide (AP).

7. The method of claim 1 wherein said polypeptides comprising a disintegrin domain is a modified ADAM-derived polypeptide (MAP).

8. The method of claim 7 wherein said polypeptides comprising a disintegrin domain is selected from the group consisting of: MAP1, MAP2, MAP3, MAP6, MAP7, MAPS, MAP9, MAP10, MAP11, MAP12, MAP15, MAP17, MAP18, MAP19, MAP20, MAP21, MAP22, MAP23, MAP28, MAP29, MAP30, MAP32, and MAP33.

9. The method of claim 1 wherein said soft tissue is in the eye.

10. The method of claim 9 wherein said soft tissue is in the retina or conjunctiva.

11. The method of claim 9 wherein said soft tissue is in the cornea or sclera.

12. The method of claim 9 wherein said soft tissue is in the lens or choroid.

13. The method of claim 1 wherein said soft tissue is in liver or brain cortex.

14. The method of claim 1 wherein said patch is reversibly bound to the soft tissue injury site.

15. The method of claim 1 wherein said patch further comprises a drug to be applied topically to the wound.

16. A method for growing cells, comprising contacting the cells with a patch comprising a polymer, said polymer being present on a surface of said patch, said polymer of said surface linked to polypeptides comprising a disintegrin domain wherein said polypeptides facilitate attachment of the cells to the patch, thereby aiding growth of the cells.

17. The method of claim 16 wherein said polymer comprises silicone and said silicone of said surface is activated.

18. The method of claim 17 wherein at least part of said silicone of said surface is activated by irradiation with laser light at a wavelength and power sufficient to eject organic species from the silicone substrate.

19. The method of claim 16 wherein said polymer comprises a parylene.

20. The method of claim 16 wherein said polypeptides comprising a disintegrin domain is contortrostatin (CN) or vicrostatin (VCN).

21. The method of claim 16 wherein said polypeptides comprising a disintegrin domain is an ADAM-derived polypeptide (AP).

22. The method of claim 16 wherein said polypeptides comprising a disintegrin domain is a modified ADAM-derived polypeptide (MAP).

23. The method of claim 22 wherein said polypeptides comprising a disintegrin domain is selected from the group consisting of: MAP1, MAP2, MAP3, MAP6, MAP7, MAPS, MAP9, MAP10, MAP11, MAP12, MAP15, MAP17, MAP18, MAP19, MAP20, MAP21, MAP22, MAP23, MAP28, MAP29, MAP30, MAP32, and MAP33.

24. The method of claim 16 wherein said cells are stem cells.

25. The method of claim 24 wherein said cells are embryonic progenitor neurons.


SEQUENCE LISTING (150 sequences)

SEQ ID NO: 1 - protein, 483 aa, Agkistrodon contortrix
MIQVLLVTLCLAAFPYQGSSIILESGNVNDYEVLYPQKVTALPKGAVQPKYEDTMQYEFKVNGEPVVLHLEKNKGLFSKDYSETHYSSDGRKITTNPPVEDHCYYHGRIQNDADSTASISACNGLKGHFKLQGETYLIEPLKLSDSEAHAVYKYENVEKEDEAPKMCGVTQTNWESDEPIKKASQLNLTPEQQGFPQRYIELVVVADHRMFTKYNGNLNTIRIWVHELVNTMNVFYRPLNIRVSLTDLEVWSDQDLINVQPAAADTLEAFGDWRETVLLNRISHDNAQLLTAIELDGETIGLANRGTMCDPKLSTGIVQDHSAINLWVAVTMAHEMGHNLGISHDGNQCHCDANSCIMSEELREQLSFEFSDCSQNQYQTYLTDHNPQCMLNEPLRTDIVSTPVSGNELLETGEESDFDAPANPCCDAATCKLTTGSQCADGLCCDQCKFMKEGTVCRRARGDDLDDYCNGISAGCPRNPFHA

SEQ ID NO: 2 - protein, 65 aa, Agkistrodon contortrix
DAPANPCCDAATCKLTTGSQCADGLCCDQCKFMKEGTVCRRARGDDLDDYCNGISAGCPRNPFHA

SEQ ID NO: 3 - protein, 69 aa, Agkistrodon contortrix
GDAPANPCCDAATCKLTTGSQCADGLCCDQCKFMKEGTVCRRARGDDLDDYCNGISAGCPRNPHKGPAT

SEQ ID NO: 4 - protein, 78 aa, Crotalus sp.
GEECDCGSPRTCRDPCCDATTCKLRQGAQCAEGLCCDQCRFKGAGTECRAAKDECDMADVCTGRSAECTDRFQRNGQP

SEQ ID NO: 5 - protein, 78 aa, Crotalus sp.
GEECDCGTPENCQNECCDAATCKLKSGSQCGHGDCCEQCKFSKSGTECRASMSECDPAEHCTGQSSECPADVFHKNGQ

SEQ ID NO: 6 - protein, 83 aa, Gloydius sp.
SPPVCGNYYPEVGEDCDCGPPANCQNPCCDAATCGLTTGSQCAEGLCCDQCRLKKAGTICRKARGDNPDDRCTGQSGVCPRNT

SEQ ID NO: 7 - protein, 83 aa, Bitis sp.
SPPVCGNKILEEGEECDCGSPANCQDRCCNAATCKLTPGSQCNHGECCDQCKFKKAGTVCRIARGDWNDDYCTGKSSDCPWNH

SEQ ID NO: 8 - protein, 70 aa, Trimeresurus sp.
GEECDCGSPSNPCCDAATCKLRPGAQCADGLCCDQCRFKKKRTICRIARGDFPDDRCTGQSADCPRWNDL

SEQ ID NO: 9 - protein, 78 aa, Homo sapiens
GEECDCGPAQECTNPCCDAHTCVLKPGFTCAEGECCESCQIKKAGSICRPAKDECDFPEMCTGHSPACPKDQFRVNGF

SEQ ID NO: 10 - protein, 78 aa, Homo sapiens
GEQCDCGPPEDCRNRCCNSTTCQLAEGAQCAHGTCCQECKVKPAGELCRPKKDMCDLEEFCDGRHPECPEDAFQENGT

SEQ ID NO: 11 - protein, 78 aa, Homo sapiens
GEECDCGEPEECMNRCCNATTCTLKPDAVCAHGLCCEDCQLKPAGTACRDSSNSCDLPEFCTGASPHCPANVYLHDGH

SEQ ID NO: 12 - protein, 78 aa, Homo sapiens
GEECDCGEEEECNNPCCNASNCTLRPGAECAHGSCCHQCKLLAPGTLCREQARQCDLPEFCTGKSPHCPTNFYQMDGT

SEQ ID NO: 13 - protein, 78 aa, Homo sapiens
GEDCDCGTSEECTNICCDAKTCKIKATFQCALGECCEKCQFKKAGMVCRPAKDECDLPEMCNGKSGNCPDDRFQVNGF

SEQ ID NO: 14 - protein, 78 aa, Homo sapiens
GEECDCGPGQECRDLCCFAHNCSLRPGAQCAHGDCCVRCLLKPAGALCRQAMGDCDLPEFCTGTSSHCPPDVYLLDGS

SEQ ID NO: 15 - protein, 76 aa, Homo sapiens
TEQCDCGSLCQHHACCDENCILKAKAECSDGPCCHKCKFHRKGYPCCPSSRSCDLPEFCNGTSALCPNNRHKQDGS

SEQ ID NO: 16 - protein, 81 aa, Homo sapiens
GEECDCGTEQDCALIGETCCDIATCRFKAGSNCAEGPCCENCLFMSKERMCRPSFEECDLPEYCNGSSASCPENHYVQTGH

SEQ ID NO: 17 - protein, 81 aa, Homo sapiens
TEQCDCGYKEACTHKKCCNPADCTLVRSAECGTGSCCNNKTCTIHERGHVCRKSVDMCDFPEYCNGTSEFCVPDVKAADLE

SEQ ID NO: 18 - protein, 78 aa, Homo sapiens
REECDCGSFKQCYASYCCQSDCHLTPGSICHIGECCTNCSFSPPGTLCRPIQNICDLPEYCHGTTVTCPANVYMQDGT

SEQ ID NO: 19 - protein, 78 aa, Homo sapiens; identical to SEQ ID NO: 9

SEQ ID NO: 20 - protein, 78 aa, Homo sapiens; identical to SEQ ID NO: 10

SEQ ID NO: 21 - protein, 79 aa, Homo sapiens
GEECDCGTPKECELDPCCEGSTCKLKSFAECAYGDCCKDCRFLPGGTLCRGKTSECDVPEYCNGSSQFCQPDVFIQNGY

SEQ ID NO: 22 - protein, 86 aa, Homo sapiens
GEECDCGYSDQCKDECCFDANQPEGRKCKLKPGKQCSPSQGPCCTAQCAFKSKSEKCRDDSDCAREGICNGFTALCPASDPKPNFT

SEQ ID NO: 23 - protein, 79 aa, Homo sapiens
GEECDCGSVQECSRAGGNCCKKCTLTHDAMCSDGLCCRRCKYEPRGVSCREAVNECDIAETCTGDSSQCPPNLHKLDGY

SEQ ID NO: 24 - protein, 78 aa, Homo sapiens; identical to SEQ ID NO: 11

SEQ ID NO: 25 - protein, 79 aa, Homo sapiens
GEQCDCGFLDDCVDPCCDSLTCQLRPGAQCASDGPCCQNCQLRPSGWQCRPTRGDCDLPEFCPGDSSQCPPDVSLGDGE

SEQ ID NO: 26 - protein, 80 aa, Homo sapiens
GEECDPGIMYLNNDTCCNSDCTLKEGVQCSDRNSPCCKNCQFETAQKKCQEAINATCKGVSYCTGNSSECPPPGNAEDDT

SEQ ID NO: 27 - protein, 81 aa, Homo sapiens
NEECDCGNKNECQFKKCCDYNTCKLKGSVKCGSGPCCTSKCELSIAGTPCRKSIDPECDFTEYCNGTSSNCVPDTYALNGR

SEQ ID NO: 28 - protein, 78 aa, Homo sapiens; identical to SEQ ID NO: 12

SEQ ID NO: 29 - protein, 78 aa, Homo sapiens
GEECDCGTIRQCAKDPCCLLNCTLHPGAACAFGICCKDCKFLPSGTLCRQQVGECDLPEWCNGTSHQCPDDVYVQDGI

SEQ ID NO: 30 - protein, 78 aa, Homo sapiens
EEQCDCGSVQQCEQDACCLLNCTLRPGAACAFGLCCKDCKFMPSGELCRQEVNECDLPEWCNGTSHQCPEDRYVQDGI

SEQ ID NO: 31 - protein, 79 aa, Homo sapiens
GEECDCGTPAECVLEGAECCKKCTLTQDSQCSDGLCCKKCKFQPMGTVCREAVNDCDIRETCSGNSSQCAPNIHKMDGY

SEQ ID NO: 32 - protein, 78 aa, Homo sapiens
GEECDCGFHVECYGLCCKKCSLSNGAHCSDGPCCNNTSCLFQPRGYECRDAVNECDITEYCTGDSGQCPPNLHKQDGY

SEQ ID NO: 33 - protein, 78 aa, Homo sapiens; identical to SEQ ID NO: 13

SEQ ID NO: 34 - protein, 78 aa, Homo sapiens
GEECDCGPLKHCAKDPCCLSNCTLTDGSTCAFGLCCKDCKFLPSGKVCRKEVNECDLPEWCNGTSHKCPDDFYVEDGI

SEQ ID NO: 35 - protein, 78 aa, Homo sapiens
NEECDCGSTEECQKDRCCQSNCKLQPGANCSIGLCCHDCRFRPSGYVCRQEGNECDLAEYCDGNSSSCPNDVYKQDGT

SEQ ID NO: 36 - protein, 80 aa, Homo sapiens
NEICDCGTEAQCGPASCCDFRTCVLKDGAKCYKGLCCKDCQILQSGVECRPKAHPECDIAENCNGSSPECGPDITLINGL

SEQ ID NO: 37 - protein, 78 aa, Homo sapiens; identical to SEQ ID NO: 14

SEQ ID NO: 38 - protein, 70 aa, Trimeresurus sp.; identical to SEQ ID NO: 8

SEQ ID NO: 39 - protein, 72 aa, Artificial Sequence
  Description of Artificial Sequence Synthetic polypeptide
TEQCDCGSLHACCDENCILKAKAECSDGPCCHKCKFHRKGYPCRPSSRSDLPEFCNGTSALCPNNRHKQDGS

SEQ ID NO: 40 - protein, 77 aa, Artificial Sequence
  Description of Artificial Sequence Synthetic polypeptide
GEECDCGTEQDIGETCCDIATCRFKAGSNCAEGPCCENCLFMSKERMCRPSFEEDLPEYCNGSSASCPENHYVQTGH

SEQ ID NO: 41 - protein, 77 aa, Artificial Sequence
  Description of Artificial Sequence Synthetic polypeptide
TEQCDCGYKEAKKCCNPADCTLVRSAECGTGSCCNNKTCTIHERGHVCRKSVDMDFPEYCNGTSEFCVPDVKAADLE

SEQ ID NO: 42 - protein, 74 aa, Artificial Sequence
  Description of Artificial Sequence Synthetic polypeptide
REECDCGSFKQSYCCQSDCHLTPGSICHIGECCTNCSFSPPGTLCRPIQNIDLPEYCHGTTVTCPANVYMQDGT

SEQ ID NO: 43 - protein, 74 aa, Artificial Sequence
  Description of Artificial Sequence Synthetic polypeptide
GEECDCGPAQEPCCDAHTCVLKPGFTCAEGECCESCQIKKAGSICRPAKDEDFPEMCTGHSPACPKDQFRVNGF

SEQ ID NO: 44 - protein, 74 aa, Artificial Sequence
  Description of Artificial Sequence Synthetic polypeptide
GEQCDCGPPEDRCCNSTTCQLAEGAQCAHGTCCQECKVKPAGELCRPKKDMDLEEFCDGRHPECPEDAFQENGT

SEQ ID NO: 45 - protein, 75 aa, Artificial Sequence
  Description of Artificial Sequence Synthetic polypeptide
GEECDCGTPKEDPCCEGSTCKLKSFAECAYGDCCKDCRFLPGGTLCRGKTSEDVPEYCNGSSQFCQPDVFIQNGY

SEQ ID NO: 46 - protein, 75 aa, Artificial Sequence
  Description of Artificial Sequence Synthetic polypeptide
GEECDCGSVQEAGGNCCKKCTLTHDAMCSDGLCCRRCKYEPRGVSCREAVNEDIAETCTGDSSQCPPNLHKLDGY

SEQ ID NO: 47 - protein, 74 aa, Artificial Sequence
  Description of Artificial Sequence Synthetic polypeptide
GEECDCGEPEERCCNATTCTLKPDAVCAHGLCCEDCQLKPAGTACRDSSNSDLPEFCTGASPHCPANVYLHDGH

SEQ ID NO: 48 - protein, 75 aa, Artificial Sequence
  Description of Artificial Sequence Synthetic polypeptide
GEQCDCGFLDDPCCDSLTCQLRPGAQCASDGPCCQNCQLRPSGWQCRPTRGDDLPEFCPGDSSQCPPDVSLGDGE

SEQ ID NO: 49 - protein, 74 aa, Artificial Sequence
  Description of Artificial Sequence Synthetic polypeptide
GEECDCGEEEEPCCNASNCTLRPGAECAHGSCCHQCKLLAPGTLCREQARQDLPEFCTGKSPHCPTNFYQMDGT

SEQ ID NO: 50 - protein, 74 aa, Artificial Sequence
  Description of Artificial Sequence Synthetic polypeptide
GEECDCGTIRQDPCCLLNCTLHPGAACAFGICCKDCKFLPSGTLCRQQVGEDLPEWCNGTSHQCPDDVYVQDGI

SEQ ID NO: 51 - protein, 74 aa, Artificial Sequence
  Description of Artificial Sequence Synthetic polypeptide
EEQCDCGSVQQDACCLLNCTLRPGAACAFGLCCKDCKFMPSGELCRQEVNEDLPEWCNGTSHQCPEDRYVQDGI

SEQ ID NO: 52 - protein, 75 aa, Artificial Sequence
  Description of Artificial Sequence Synthetic polypeptide
GEECDCGTPAEEGAECCKKCTLTQDSQCSDGLCCKKCKFQPMGTVCREAVNDDIRETCSGNSSQCAPNIHKMDGY

SEQ ID NO: 53 - protein, 74 aa, Artificial Sequence
  Description of Artificial Sequence Synthetic polypeptide
GEECDCGFHVELCCKKCSLSNGAHCSDGPCCNNTSCLFQPRGYECRDAVNEDITEYCTGDSGQCPPNLHKQDGY

SEQ ID NO: 54 - protein, 74 aa, Artificial Sequence
  Description of Artificial Sequence Synthetic polypeptide
GEDCDCGTSEEICCDAKTCKIKATFQCALGECCEKCQFKKAGMVCRPAKDEDLPEMCNGKSGNCPDDRFQVNGF

SEQ ID NO: 55 - protein, 74 aa, Artificial Sequence
  Description of Artificial Sequence Synthetic polypeptide
GEECDCGPLKHDPCCLSNCTLTDGSTCAFGLCCKDCKFLPSGKVCRKEVNEDLPEWCNGTSHKCPDDFYVEDGI

SEQ ID NO: 56 - protein, 74 aa, Artificial Sequence
  Description of Artificial Sequence Synthetic polypeptide
NEECDCGSTEEDRCCQSNCKLQPGANCSIGLCCHDCRFRPSGYVCRQEGNEDLAEYCDGNSSSCPNDVYKQDGT

SEQ ID NO: 57 - protein, 74 aa, Artificial Sequence
  Description of Artificial Sequence Synthetic polypeptide
GEECDCGPGQELCCFAHNCSLRPGAQCAHGDCCVRCLLKPAGALCRQAMGDDLPEFCTGTSSHCPPDVYLLDGS

SEQ ID NO: 58 - DNA, 216 bp, Artificial Sequence
  Description of Artificial Sequence Synthetic polynucleotide
acggagcagtgtgactgtggttctctacatgcatgctgtgatgaaaactgtatactgaaggcgaaagcagagtgcagtgatggtccatgttgtcataagtgtaaatttcaccgtaagggatatccttgcaggccttctagtcgttccgatctcccagaattttgcaatggtacatctgcattatgccccaacaacaggcataagcaagatggctca

SEQ ID NO: 59 - DNA, 231 bp, Artificial Sequence
  Description of Artificial Sequence Synthetic polynucleotide
ggagaggagtgtgactgtgggactgaacaggatattggagaaacatgctgtgatattgccacatgtagatttaaagccggttcaaactgtgctgaaggaccatgctgcgaaaactgtctatttatgtcaaaagaaagaatgtgtaggccttcctttgaagaagacctccctgaatattgcaatggatcatctgcatcatgcccagaaaaccactatgttcagactgggcat

SEQ ID NO: 60 - DNA, 231 bp, Artificial Sequence
  Description of Artificial Sequence Synthetic polynucleotide
acagagcagtgtgattgtggctataaagaggcaaaaaaatgctgtaatcctgcagattgtactctagttagatctgcagaatgtggcactggatcatgctgtaacaataaaacttgtacgatccacgaaagaggccatgtctgcagaaaaagtgtagatatggattttccagaatattgcaatggaacatctgagttttgtgtacctgatgtgaaagctgctgatttagaa

SEQ ID NO: 61 - DNA, 222 bp, Artificial Sequence
  Description of Artificial Sequence Synthetic polynucleotide
agggaggaatgtgactgtggctccttcaagcagagttattgctgccaaagtgactgtcacttaacaccggggagcatctgccatataggagagtgctgtacaaactgcagcttctccccaccagggactctctgcagacctatccaaaatatagaccttccagagtactgtcacgggaccaccgtgacatgtcccgcaaacgtttatatgcaagatggaacc

SEQ ID NO: 62 - DNA, 222 bp, Artificial Sequence
  Description of Artificial Sequence Synthetic polynucleotide
ggtgaagagtgtgactgtggccctgctcaggagccttgctgtgatgcacacacatgtgtactgaagccaggatttacttgtgcagaaggagaatgctgtgaatcttgtcagataaaaaaagcagggtccatatgcagaccggcgaaagatgaagattttcctgagatgtgcactggccactcgcctgcctgtcctaaggaccagttcagggtcaatggattt

SEQ ID NO: 63 - DNA, 222 bp, Artificial Sequence
  Description of Artificial Sequence Synthetic polynucleotide
ggggagcagtgcgactgcggcccccccgaggaccgctgctgcaactctaccacctgccagctggctgagggggcccagtgtgcgcacggtacctgctgccaggagtgcaaggtgaagccggctggtgagctgtgccgtcccaagaaggacatggacctcgaggagttctgtgacggccggcaccctgagtgcccggaagacgccttccaggagaacggcacg

SEQ ID NO: 64 - DNA, 225 bp, Artificial Sequence
  Description of Artificial Sequence Synthetic polynucleotide
ggggaagagtgtgactgtggtactccaaaggaagacccttgctgcgaaggaagtacctgtaagcttaaatcatttgctgagtgtgcatatggtgactgttgtaaagactgtcggttccttccaggaggtactttatgccgaggaaaaaccagtgaggatgttccagagtactgcaatggttcttctcagttctgtcagccagatgtttttattcagaatggatat

SEQ ID NO: 65 - DNA, 246 bp, Artificial Sequence
  Description of Artificial Sequence Synthetic polynucleotide
ggtgaagaatgtgattgtggctatagtgaccaggaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagacgcaagggaaggaatatgtaatggcttcacagctctctgcccagcatctgaccctaaaccaaacttcaca

SEQ ID NO: 66 - DNA, 225 bp, Artificial Sequence
  Description of Artificial Sequence Synthetic polynucleotide
ggggaggagtgcgactgcggctcggtgcaggaggcaggtggcaactgctgcaagaaatgcaccctgactcacgacgccatgtgcagcgacgggctctgctgtcgccgctgcaagtacgaaccacggggtgtgtcctgccgagaggccgtgaacgaggacatcgcggagacctgcaccggggactctagccagtgcccgcctaacctgcacaagctggacggttac

SEQ ID NO: 67 - DNA, 222 bp, Artificial Sequence
  Description of Artificial Sequence Synthetic polynucleotide
ggagaggagtgtgactgtggggagccagaggaacgctgctgcaatgccaccacctgtaccctgaagccggacgctgtgtgcgcacatgggctgtgctgtgaagactgccagctgaagcctgcaggaacagcgtgcagggactccagcaactccgacctcccagagttctgcacaggggccagccctcactgcccagccaacgtgtacctgcacgatgggcac

SEQ ID NO: 68 - DNA, 225 bp, Artificial Sequence
  Description of Artificial Sequence Synthetic polynucleotide
ggcgagcagtgtgactgtggcttcctggatgacccctgctgtgattctttgacctgccagctgaggccaggtgcacagtgtgcatctgacggaccctgttgtcaaaattgccagctgcgcccgtctggctggcagtgtcgtcctaccagaggggatgacttgcctgaattctgcccaggagacagctcccagtgtccccctgatgtcagcctaggggatggcgag

SEQ ID NO: 69 - DNA, 231 bp, Artificial Sequence
  Description of Artificial Sequence Synthetic polynucleotide
ggagaagagtgtgattgtggcatcatgtatctggacacctgctgcaacagcgactgcacgttgaaggaaggtgtccagtgcagtgacaggaacagtccttgctgtaaaaactgtcagtttgagactgcccagaagaagtgccaggaggcgattaatgctactaaaggcgtgtcctactgcacaggtaatagcagtgagtgcccgcctccaggaaatgctgaagatgacact

SEQ ID NO: 70 - DNA, 231 bp, Artificial Sequence
  Description of Artificial Sequence Synthetic polynucleotide
aatgaagaatgtgactgtggtaataaaaatgaaaagaagtgctgtgattataacacatgtaaactgaagggctcagtaaaatgtggttctggaccatgttgtacatcaaagtgtgagttgtcaatagcaggcactccatgtagaaagagtattgatccagaggattttacagagtactgcaatggaacctctagtaattgtgttcctgacacttatgcattgaatggccgt

SEQ ID NO: 71 - DNA, 222 bp, Artificial Sequence
  Description of Artificial Sequence Synthetic polynucleotide
ggggaagagtgtgactgtggagaagaagaggaaccctgctgcaatgcctctaattgtaccctgaggccgggggcggagtgtgctcacggctcctgctgccaccagtgtaagctgttggctcctgggaccctgtgccgcgagcaggccaggcaggacctcccggagttctgtacgggcaagtctccccactgccctaccaacttctaccagatggatggtacc

SEQ ID NO: 72 - DNA, 222 bp, Artificial Sequence
  Description of Artificial Sequence Synthetic polynucleotide
ggggaggaatgtgactgtggaaccatacggcaggatccctgttgtctgttaaactgtactctacatcctggggctgcttgtgcttttggaatatgttgcaaagactgcaaatttctgccatcaggaactttatgtagacaacaagttggtgaagaccttccagagtggtgcaatgggacatcccatcaatgcccagatgatgtgtatgtgcaggacgggatc

SEQ ID NO: 73 - DNA, 222 bp, Artificial Sequence
  Description of Artificial Sequence Synthetic polynucleotide
gaagagcagtgtgactgtggatccgtacagcaggacgcctgttgtctgttgaactgcactctaaggcctggggctgcctgtgcttttgggctttgttgcaaagactgcaagttcatgccatcaggggaactctgtagacaagaggtcaatgaagaccttccagaatggtgcaatggaacatctcatcagtgtccagaagatagatatgtgcaggacgggatc

SEQ ID NO: 74 - DNA, 225 bp, Artificial Sequence
  Description of Artificial Sequence Synthetic polynucleotide
ggagaggagtgtgattgtggaaccccggccgaagaaggagcagagtgttgtaagaaatgcaccttgactcaagactctcaatgcagtgacggtctttgctgtaaaaagtgcaagtttcagcctatgggcactgtgtgccgagaagcagtaaatgatgatattcgtgaaacgtgctcaggaaattcaagccagtgtgcccctaatattcataaaatggatggatat

SEQ ID NO: 75 - DNA, 222 bp, Artificial Sequence
  Description of Artificial Sequence Synthetic polynucleotide
ggggaggagtgtgattgtggttttcatgtggaattatgctgtaagaaatgttccctctccaacggggctcactgcagcgacgggccctgctgtaacaatacctcatgtctttttcagccacgagggtatgaatgccgggatgctgtgaacgaggatattactgaatattgtactggagactctggtcagtgcccaccaaatcttcataagcaagacggatat

SEQ ID NO: 76 - DNA, 222 bp, Artificial Sequence
  Description of Artificial Sequence Synthetic polynucleotide
ggagaggactgtgattgtgggacatctgaggaaatttgctgtgatgctaagacatgtaaaatcaaagcaacttttcaatgtgcattaggagaatgttgtgaaaaatgccaatttaaaaaggctgggatggtgtgcagaccagcaaaagatgaggacctgcctgaaatgtgtaatggtaaatctggtaattgtcctgatgatagattccaagtcaatggcttc

SEQ ID NO: 77 - DNA, 222 bp, Artificial Sequence
  Description of Artificial Sequence Synthetic polynucleotide
ggagaagagtgtgactgtggacctttaaagcatgatccctgctgtctgtcaaattgcactctgactgatggttctacttgtgcttttgggctttgttgcaaagactgcaagttcctaccatcagggaaagtgtgtagaaaggaggtcaatgaagatcttccagagtggtgcaatggtacttcccataagtgcccagatgacttttatgtggaagatggaatt

SEQ ID NO: 78 - DNA, 222 bp, Artificial Sequence
  Description of Artificial Sequence Synthetic polynucleotide
aatgaggaatgtgactgtggttccacagaggaggatcggtgttgccaatcaaattgtaagttgcaaccaggtgccaactgtagcattggactttgctgtcatgattgtcggtttcgtccatctggatacgtgtgtaggcaggaaggaaatgaagaccttgcagagtactgcgacgggaattcaagttcctgcccaaatgacgtttataagcaggatggaacc

SEQ ID NO: 79 - DNA, 228 bp, Artificial Sequence
  Description of Artificial Sequence Synthetic polynucleotide
aatgaaatctgtgattgtggtactgaggctcaagcaagctgttgtgattttcgaacttgtgtactgaaagacggagcaaaatgttataaaggactgtgctgcaaagactgtcaaattttacaatcaggcgttgaatgtaggccgaaagcacatcctgaagacatcgctgaaaattgtaatggaagctcaccagaatgtggtcctgacataactttaatcaatggactt

SEQ ID NO: 80 - DNA, 222 bp, Artificial Sequence
  Description of Artificial Sequence Synthetic polynucleotide
ggcgaggagtgtgactgcggccctggccaggagctctgctgctttgctcacaactgctcgctgcgcccgggggcccagtgcgcccacggggactgctgcgtgcgctgcctgctgaagccggctggagcgctgtgccgccaggccatgggtgacgacctccctgagttttgcacgggcacctcctcccactgtcccccagacgtttacctactggacggctca

SEQ ID NO: 81 - DNA, 57 bp, Artificial Sequence
  Description of Artificial Sequence Synthetic primer
gttccagatctcgaaaacctgtattttcagggcacggagcagtgtgactgtggttct

SEQ ID NO: 82 - DNA, 45 bp, Artificial Sequence
  Description of Artificial Sequence Synthetic primer
gttattcgccatggcttatgagccatcttgcttatgcctgttgtt

SEQ ID NO: 83 - DNA, 57 bp, Artificial Sequence
  Description of Artificial Sequence Synthetic primer
gttccagatctcgaaaacctgtattttcagggagaggagtgtgactgtgggactgaa

SEQ ID NO: 84 - DNA, 45 bp, Artificial Sequence
  Description of Artificial Sequence Synthetic primer
gttattcgccatggcttaatgcccagtctgaacatagtggttttc

SEQ ID NO: 85 - DNA, 59 bp, Artificial Sequence
  Description of Artificial Sequence Synthetic primer
gttccgccatggctgaaaacctgtattttcagggtacagagcagtgtgattgtggctat

SEQ ID NO: 86 - DNA, 44 bp, Artificial Sequence
  Description of Artificial Sequence Synthetic primer
gttattcggaattcttattctaaatcagcagctttcacatcagg

SEQ ID NO: 87 - DNA, 57 bp, Artificial Sequence
  Description of Artificial Sequence Synthetic primer
gttccagatctcgaaaacctgtattttcagggcagggaggaatgtgactgtggctcc

SEQ ID NO: 88 - DNA, 45 bp, Artificial Sequence
  Description of Artificial Sequence Synthetic primer
gttattcgccatggcttaggttccatcttgcatataaacgtttgc

SEQ ID NO: 89 - DNA, 57 bp, Artificial Sequence
  Description of Artificial Sequence Synthetic primer
gttccagatctcgaaaacctgtattttcagggtgaagagtgtgactgtggccctgct

SEQ ID NO: 90 - DNA, 45 bp, Artificial Sequence
  Description of Artificial Sequence Synthetic primer
gttattcgccatggcttaaaatccattgaccctgaactggtcctt

SEQ ID NO: 91 - DNA, 57 bp, Artificial Sequence
  Description of Artificial Sequence Synthetic primer
gttccagatctcgaaaacctgtattttcagggggagcagtgcgactgcggccccccc

SEQ ID NO: 92 - DNA, 45 bp, Artificial Sequence
  Description of Artificial Sequence Synthetic primer
gttattcgccatggcttacgtgccgttctcctggaaggcgtcttc

SEQ ID NO: 93 - DNA, 57 bp, Artificial Sequence
  Description of Artificial Sequence Synthetic primer
gttccagatctcgaaaacctgtattttcagggggaagagtgtgactgtggtactcca

SEQ ID NO: 94 - DNA, 45 bp, Artificial Sequence
  Description of Artificial Sequence Synthetic primer
gttattcgccatggcttaatatccattctgaataaaaacatctgg

SEQ ID NO: 95 - DNA, 57 bp, Artificial Sequence
  Description of Artificial Sequence Synthetic primer
gttccagatctcgaaaacctgtattttcagggtgaagaatgtgattgtggctatagt

SEQ ID NO: 96 - DNA, 45 bp, Artificial Sequence
  Description of Artificial Sequence Synthetic primer
gttattcgccatggcttatgtgaagtttggtttagggtcagatgc

SEQ ID NO: 97 - DNA, 57 bp, Artificial Sequence
  Description of Artificial Sequence Synthetic primer
gttccagatctcgaaaacctgtattttcagggggaggagtgcgactgcggctcggtg

SEQ ID NO: 98 - DNA, 45 bp, Artificial Sequence
  Description of Artificial Sequence Synthetic primer
gttattcgccatggcttagtaaccgtccagcttgtgcaggttagg

SEQ ID NO: 99 - DNA, 57 bp, Artificial Sequence
  Description of Artificial Sequence Synthetic primer
gttccagatctcgaaaacctgtattttcagggagaggagtgtgactgtggggagcca

SEQ ID NO: 100 - DNA, 45 bp, Artificial Sequence
  Description of Artificial Sequence Synthetic primer
gttattcgccatggcttagtgcccatcgtgcaggtacacgttggc

SEQ ID NO: 101 - DNA, 57 bp, Artificial Sequence
  Description of Artificial Sequence Synthetic primer
gttccagatctcgaaaacctgtattttcagggcgagcagtgtgactgtggcttcctg

SEQ ID NO: 102 - DNA, 45 bp, Artificial Sequence
  Description of Artificial Sequence Synthetic primer
gttattcgccatggcttactcgccatcccctaggctgacatcagg

SEQ ID NO: 103 - DNA, 57 bp, Artificial Sequence
  Description of Artificial Sequence Synthetic primer
gttccagatctcgaaaacctgtattttcagggagaagagtgtgattgtggcatcatg

SEQ ID NO: 104 - DNA, 45 bp, Artificial Sequence
  Description of Artificial Sequence Synthetic primer
gttattcgccatggcttaagtgtcatcttcagcatttcctggagg

SEQ ID NO: 105 - DNA, 57 bp, Artificial Sequence
  Description of Artificial Sequence Synthetic primer
gttccagatctcgaaaacctgtattttcagggcaatgaagaatgtgactgtggtaat

SEQ ID NO: 106 - DNA, 45 bp, Artificial Sequence
  Description of Artificial Sequence Synthetic primer
gttattcgccatggcttaacggccattcaatgcataagtgtcagg

SEQ ID NO: 107 - DNA, 57 bp, Artificial Sequence
  Description of Artificial Sequence Synthetic primer
gttccagatctcgaaaacctgtattttcagggggaagagtgtgactgtggagaagaa

SEQ ID NO: 108 - DNA, 45 bp, Artificial Sequence
  Description of Artificial Sequence Synthetic primer
gttattcgccatggcttaggtaccatccatctggtagaagttggt

SEQ ID NO: 109 - DNA, 57 bp, Artificial Sequence
  Description of Artificial Sequence Synthetic primer
gttccagatctcgaaaacctgtattttcagggggaggaatgtgactgtggaaccata

SEQ ID NO: 110 - DNA, 45 bp, Artificial Sequence
  Description of Artificial Sequence Synthetic primer
gttattcgccatggcttagatcccgtcctgcacatacacatcatc

SEQ ID NO: 111 - DNA, 57 bp, Artificial Sequence
  Description of Artificial Sequence Synthetic primer
gttccagatctcgaaaacctgtattttcagggcgaagagcagtgtgactgtggatcc

SEQ ID NO: 112 - DNA, 45 bp, Artificial Sequence
  Description of Artificial Sequence Synthetic primer
gttattcgccatggcttagatcccgtcctgcacatatctatcttc

SEQ ID NO: 113 - DNA, 57 bp, Artificial Sequence
  Description of Artificial Sequence Synthetic primer
gttccagatctcgaaaacctgtattttcagggagaggagtgtgattgtggaaccccg

SEQ ID NO: 114 - DNA, 45 bp, Artificial Sequence
  Description of Artificial Sequence Synthetic primer
gttattcgccatggcttaatatccatccattttatgaatattagg

SEQ ID NO: 115 - DNA, 57 bp, Artificial Sequence
  Description of Artificial Sequence Synthetic primer
gttccagatctcgaaaacctgtattttcagggggaggagtgtgattgtggttttcat

SEQ ID NO: 116 - DNA, 45 bp, Artificial Sequence
  Description of Artificial Sequence Synthetic primer
gttattcgccatggcttaatatccgtcttgcttatgaagatttgg

SEQ ID NO: 117 - DNA, 57 bp, Artificial Sequence
  Description of Artificial Sequence Synthetic primer
gttccagatctcgaaaacctgtattttcagggagaggactgtgattgtgggacatct

SEQ ID NO: 118 - DNA, 45 bp, Artificial Sequence
  Description of Artificial Sequence Synthetic primer
gttattcgccatggcttagaagccattgacttggaatctatcatc

SEQ ID NO: 119 - DNA, 59 bp, Artificial Sequence
  Description of Artificial Sequence Synthetic primer
gttccgccatggctgaaaacctgtattttcagggagaagagtgtgactgtggaccttta

SEQ ID NO: 120 - DNA, 44 bp, Artificial Sequence
  Description of Artificial Sequence Synthetic primer
gttattcggaattcttaaattccatcttccacataaaagtcatc

SEQ ID NO: 121 - DNA, 57 bp, Artificial Sequence
  Description of Artificial Sequence Synthetic primer
gttccagatctcgaaaacctgtattttcagggcaatgaggaatgtgactgtggttcc

SEQ ID NO: 122 - DNA, 45 bp, Artificial Sequence
  Description of Artificial Sequence Synthetic primer
gttattcgccatggcttaggttccatcctgcttataaacgtcatt

SEQ ID NO: 123 - DNA, 57 bp, Artificial Sequence
  Description of Artificial Sequence Synthetic primer
gttccagatctcgaaaacctgtattttcagggcaatgaaatctgtgattgtggtact

SEQ ID NO: 124 - DNA, 45 bp, Artificial Sequence
  Description of Artificial Sequence Synthetic primer
gttattcgccatggcttaaagtccattgattaaagttatgtcagg

SEQ ID NO: 125 - DNA, 57 bp, Artificial Sequence
  Description of Artificial Sequence Synthetic primer
gttccagatctcgaaaacctgtattttcagggcgaggagtgtgactgcggccctggc

SEQ ID NO: 126 - DNA, 45 bp, Artificial Sequence
  Description of Artificial Sequence Synthetic primer
gttattcggaattccttatgagccgtccagtaggtaaacgtctgg

SEQ ID NO: 127 - protein, 230 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGTEQCDCGSLHACCDENCILKAKAECSDGPCCHKCKFHRKGYPCRPSSRSDLPEFCNGTSALCPNNRHKQDGS

SEQ ID NO: 128 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTEQDIGETCCDIATCRFKAGSNCAEGPCCENCLFMSKERMCRPSFEEDLPEYCNGSSASCPENHYVQTGH

SEQ ID NO: 129 - protein, 245 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAENLYFQGTEQCDCGYKEAKKCCNPADCTLVRSAECGTGSCCNNKTCTIHERGHVCRKSVDMDFPEYCNGTSEFCVPDVKAADLE

SEQ ID NO: 130 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGREECDCGSFKQSYCCQSDCHLTPGSICHIGECCTNCSFSPPGTLCRPIQNIDLPEYCHGTTVTCPANVYMQDGT

SEQ ID NO: 131 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGPAQEPCCDAHTCVLKPGFTCAEGECCESCQIKKAGSICRPAKDEDFPEMCTGHSPACPKDQFRVNGF

SEQ ID NO: 132 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGPPEDRCCNSTTCQLAEGAQCAHGTCCQECKVKPAGELCRPKKDMDLEEFCDGRHPECPEDAFQENGT

SEQ ID NO: 133 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTPKEDPCCEGSTCKLKSFAECAYGDCCKDCRFLPGGTLCRGKTSEDVPEYCNGSSQFCQPDVFIQNGY

SEQ ID NO: 134 - protein, 239 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGYSDQECCFDANQPEGRKCKLKPGKQCSPSQGPCCTAQCAFKSKSEKCRDDSDAREGICNGFTALCPASDPKPNFT

SEQ ID NO: 135 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGSVQEAGGNCCKKCTLTHDAMCSDGLCCRRCKYEPRGVSCREAVNEDIAETCTGDSSQCPPNLHKLDGY

SEQ ID NO: 136 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGEPEERCCNATTCTLKPDAVCAHGLCCEDCQLKPAGTACRDSSNSDLPEFCTGASPHCPANVYLHDGH

SEQ ID NO: 137 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGFLDDPCCDSLTCQLRPGAQCASDGPCCQNCQLRPSGWQCRPTRGDDLPEFCPGDSSQCPPDVSLGDGE

SEQ ID NO: 138 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGIMYLDTCCNSDCTLKEGVQCSDRNSPCCKNCQFETAQKKCQEAINATKGVSYCTGNSSECPPPGNAEDDT

SEQ ID NO: 139 - protein, 235 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGNEECDCGNKNEKKCCDYNTCKLKGSVKCGSGPCCTSKCELSIAGTPCRKSIDPEDFTEYCNGTSSNCVPDTYALNGR

SEQ ID NO: 140 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGEEEEPCCNASNCTLRPGAECAHGSCCHQCKLLAPGTLCREQARQDLPEFCTGKSPHCPTNFYQMDGT

SEQ ID NO: 141 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTIRQDPCCLLNCTLHPGAACAFGICCKDCKFLPSGTLCRQQVGEDLPEWCNGTSHQCPDDVYVQDGI

SEQ ID NO: 142 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQEEQCDCGSVQQDACCLLNCTLRPGAACAFGLCCKDCKFMPSGELCRQEVNEDLPEWCNGTSHQCPEDRYVQDGI

SEQ ID NO: 143 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTPAEEGAECCKKCTLTQDSQCSDGLCCKKCKFQPMGTVCREAVNDDIRETCSGNSSQCAPNIHKMDGY

SEQ ID NO: 144 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGFHVELCCKKCSLSNGAHCSDGPCCNNTSCLFQPRGYECRDAVNEDITEYCTGDSGQCPPNLHKQDGY

SEQ ID NO: 145 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEDCDCGTSEEICCDAKTCKIKATFQCALGECCEKCQFKKAGMVCRPAKDEDLPEMCNGKSGNCPDDRFQVNGF

SEQ ID NO: 146 - protein, 241 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAENLYFQGEECDCGPLKHDPCCLSNCTLTDGSTCAFGLCCKDCKFLPSGKVCRKEVNEDLPEWCNGTSHKCPDDFYVEDGI

SEQ ID NO: 147 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGNEECDCGSTEEDRCCQSNCKLQPGANCSIGLCCHDCRFRPSGYVCRQEGNEDLAEYCDGNSSSCPNDVYKQDGT

SEQ ID NO: 148 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGNEICDCGTEAQASCCDFRTCVLKDGAKCYKGLCCKDCQILQSGVECRPKAHPEDIAENCNGSSPECGPDITLINGL

SEQ ID NO: 149 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGPGQELCCFAHNCSLRPGAQCAHGDCCVRCLLKPAGALCRQAMGDDLPEFCTGTSSHCPPDVYLLDGS

SEQ ID NO: 150 - DNA, 2018 bp, Agkistrodon contortrix
attcggggtcaatagaggaagagctcaagttggcttgaaagcaggaagagattgcctgtcttccagccaaatccagccgccaaaatgatccaggttctcttggtgactctatgcttagcagcttttccttatcaagggagctctataatcctggaatctgggaatgttaatgattatgaagtactgtatccacaaaaagtcactgcattgcccaaaggagcagttcagccaaagtatgaagacaccatgcaatatgaatttaaagtgaatggagagccagtggtccttcacctggaaaaaaataaaggacttttttcaaaagattacagcgagactcattattcctctgatggcagaaaaattacaacaaaccctccggttgaggatcactgctattatcatggacgcatccagaatgatgctgactcaactgcaagcatcagtgcatgcaacggtttgaaaggacatttcaagcttcaaggggagacgtaccttattgaacccttgaagctttccgacagtgaagcccatgcagtctacaaatatgaaaacgtagaaaaagaagatgaggcccccaaaatgtgtggggtaacccagactaattgggaatcagatgagcccatcaaaaaggcctctcagttaaatcttactcctgaacaacaaggattcccccaaagatacattgagcttgttgtagttgcagatcacagaatgttcacgaaatacaacggcaatttaaatactattagaatatgggtacatgaacttgtcaacactatgaatgtgttttacagacctttgaatattcgtgtctcactgactgacctagaagtttggtcagaccaagatttgatcaacgtgcagccagcagcggctgatactttggaagcatttggagactggagagagacagtcttgctgaatcgcataagtcatgataatgctcagttactcacggccattgagcttgatggagaaactataggattggctaacaggggcaccatgtgcgacccgaagctttctacaggaattgttcaggatcatagtgcaataaatctttgggttgcagttacaatggcccatgagatgggtcataatctgggtattagtcacgatggaaatcagtgtcattgcgatgctaactcatgcattatgagtgaagaactaagagaacaactttcctttgagttcagcgattgtagtcagaatcaatatcagacatatcttactgatcataacccacaatgcatgctcaatgaacccttgagaacagatattgtttcaactccagtttctggaaatgaacttttggagacgggagaagaaagtgactttgacgctcctgcaaatccgtgctgcgatgctgcaacatgtaaactgacaacagggtcacagtgtgcagatggactgtgttgtgaccagtgcaaatttatgaaagaaggaacagtatgccggagagcaaggggtgatgacctggatgattactgcaatggcatatctgctggctgtcccagaaatcccttccatgcctaaccaacaatggagatggaatggtctgcagcaacaggcagtgtgttgatctgaatacagcctaataatcaacctctggcttctctcagatttgatcatggagatccttcttccagaaggtttcacttccctcaaatccaaagagacccatctgcctgcatcctactagtaaatcacccttagcttccagatggtatccaaattctgtaatatttcttctccatatttaatctatttaccttttgctgtaacaaaacctttttcctgtcacaaagctccatgggcatgtacagcttatctgctgtcaagaaaaaaaatggccattttaccgtttgccagttacaaagcacatttaatgcaacaagttcttccttttgagctgatgtattcaaagtcaatgcttcctctcccaaaatttcatgctggcttcccaagatgtagctgcttccgtcaataaacaaactattctcattcaaaaaaaaaaa